(19) United States
(12) Patent Application Publication
(10) Pub. No.: US 2010/0185220 A1
(43) Pub. Date: Jul. 22, 2010

(54) APPARATUS AND METHODS FOR CONTROLLED ISCHEMIC CONDITIONING

(75) Inventors: Morteza Naghavi, Houston, TX (US); Albert Andrew Yen, Houston, TX (US); Haider Hassan, Houston, TX (US); David Panthagani, Houston, TX (US)

Correspondence Address: LAW OFFICE OF DAVID MCEWING, P.O. BOX 70410, HOUSTON, TX, 77270, US

(73) Assignee: IC THERAPEUTICS, INC., Houston, TX (US)

(21) Appl. No.: 12/601,391

(22) PCT Filed: May 23, 2008
(86) PCT No.: PCT/US08/64792

§ 371 Date: Nov. 23, 2009

Related U.S. Application Data
(63) Continuation of application No. PCT/US08/64792, filed on May 23, 2008.
(60) Provisional application No. 60/939,821, filed on May 23, 2007.
(60) Provisional application No. 60/929,863, filed on Jul. 16, 2007.
(60) Provisional application No. 61/025,715, filed on Feb. 1, 2008.
(60) Provisional application No. 61/029,147, filed on Feb. 15, 2008.

(51) Int. Cl. A61B 17/00; A61B 5/00

(52) U.S. Cl. 606/158; 600/301

(57) ABSTRACT

Methods and apparatus for ischemic conditioning treatment in a patient are provided. Transient ischemia is caused by interrupting blood flow to a tissue. Markers of ischemia and metabolism are monitored in the tissue and the induced ischemia is adjusted based on the monitoring results.

### CROSS REFERENCE TO RELATED APPLICATIONS

This application claims priority to PCT/US08/64792, filed on May 23, 2008 and published as WO 2008/148062. Priority is also claimed to the applications to which priority was claimed in PCT/US08/64792, to wit, U.S. Provisional Application No. 60/939,821 filed May 23, 2007, U.S. Provisional Application No. 60/969,863 filed Sep. 4, 2007, U.S. Provisional Application No. 61/025,715 filed Feb. 1, 2008, and U.S. Provisional Application No. 61/029,147 filed Feb. 15, 2008. The disclosures of each priority claim are incorporated herein by reference in their entireties.

### BACKGROUND

The disclosures herein relate generally to apparatus and methods for reducing damage to tissues and improving response to therapies. More particularly, this invention relates to apparatus and methods for utilizing ischemic conditioning in the prevention, reduction and treatment of disease conditions.

Brief repeated periods of ischemia (a local shortage of oxygen-carrying blood supply) in biological tissue are known in some systems to render that tissue more resistant to subsequent ischemic insults. This is phenomena is known as ischemic preconditioning. Further, for an organ or tissue already undergoing total or subtotal ischemia, blood flow conditions can be modified during the onset of resumed blood flow to significantly reduce reperfusion injury. Since this method begins at the onset of resuming blood flow after ischemia, it is known as postconditioning.

Ischemic conditioning exerts tissue protection and appears to be a ubiquitous endogenous protective mechanism at the cellular level that has been observed in the heart of humans and other animal species tested. This protection has also been seen in organs such as the stomach, liver, kidney, gut, skeletal tissue, urinary bladder and brain. See D M Yellon and J M Downey, “Preconditioning the myocardium: from cellular physiology to clinical cardiology,” Physiol Rev 83 (2003) 1113-1151.

What are needed are apparatus and methods that adapt the experimental phenomena of ischemic conditioning to safe and effective therapies by providing, for the first time, individualized control and monitoring of the process.

### SUMMARY

Provided herein are apparatus and methods for utilizing ischemic conditioning by a controlled series of vascular occlusions and reperfusions of one or more blood vessels and/or vascularised body systems. The present invention is adapted to minimize damage to blood vessels while maximizing the value of the process in different individuals.

The invention can be adapted to repeated occlusion and release of the blood vessel manually and/or in accordance with an automated schedule. In certain automated embodiments, ease of use is improved by automating with a programmable controller. In an embodiment, the invention can control the one or more occlusions based on monitoring of markers of ischemia.

Suitable occluding members can be designed in numerous variations to exert force on a blood vessel. Occluding members can include but are not limited to components that are: pistoned, jawed, coiled, inflatable, clamping, steerable, ringed, timed, and/or combinations thereof. In an embodiment, a controller is adapted to position and tighten the occluding member against the blood vessel to induce at least a partial occlusion. In an embodiment, a programmable controller is employed to control the occlusion and release of the occluding member.

In one embodiment, sensors are added to the system for the detection of markers of ischemia and/or metabolism in tissues that are affected by the induced ischemia. In an embodiment, at least a portion of the occlusion and release is controlled by a programmable controller based on monitoring of the sensed markers. In further embodiments, a blood flow sensor is added at a position, relative to the flow of blood, that is distal to the occluding member.

In an embodiment, one or more occlusions and releases can be part of an intervention for induction of collaterals or angiogenesis, inducing controlled necrosis, reducing reperfusion injury, or combinations thereof.

In an embodiment, the occlusion has application to other luminal tissues in addition to blood vessels, such as for example the esophagus. In an embodiment, the procedure is performed endoscopically.

In an embodiment, more than one blood vessel can be occluded at the same time by the occluding member. In an embodiment, the occluding member can achieve occlusion through inflation, motorized methods, manual methods, automated methods, or combinations thereof. In an embodiment, the occluding member can be attached to a luminal tissue for multiple occlusions.

### BRIEF DESCRIPTION OF THE DRAWINGS

FIGS. 1A-C depict various systems for controlling extravascular occlusions.

FIGS. 2A-I depict cross-sectional views of embodiments of pistoned occluding members capable of extravascular occlusion.

FIGS. 3A-G depict cross-sectional views of embodiments of jawed occluding members capable of extravascular occlusion.

FIGS. 4A-G depict cross-sectional views of embodiments of coiled occluding members capable of extravascular occlusion.

FIGS. 5A-B depict cross-sectional views of embodiments of steerable occluding members capable of extravascular occlusion.

FIGS. 6A-J depict cross-sectional views of embodiments of inflatable occluding members capable of extravascular occlusion.

FIG. 7A depicts an embodiment of an unlocked minimally invasive cuff around a blood vessel as used for ischemic conditioning and FIG. 7B depicts an embodiment of a locked minimally invasive cuff as used for ischemic conditioning.

FIG. 8A depicts a system for external delivery of a minimally invasive cuff as used for ischemic conditioning. FIG. 8B depicts a system for internal delivery of a minimally invasive cuff as used for ischemic conditioning.

FIGS. 9A-E depict cross-sectional views of embodiments of stabilizing members that can be provided.

FIGS. 10A-C depict embodiments of orientations of stabilizing members relative to occluding members.

FIGS. 11A-D depict various systems for extravascular occlusion.

FIG. 12 depicts embodiments of motored mechanisms that can provide controlled force for one or more extravascular occlusions.

FIG. 13 depicts a motorized system for extravascular occlusion.

FIG. 14 depicts a system for performing multiple extravascular occlusions on multiple sites simultaneously.

FIG. 15 depicts a system for ischemic conditioning.

FIGS. 16A-C depict several systems for ischemic conditioning.

FIG. 17A depicts an embodiment of a minimally invasive cuff deflated around a blood vessel as used for ischemic conditioning. FIG. 17B depicts an embodiment of a minimally invasive cuff inflated around a blood vessel as used for ischemic conditioning.

FIG. 18A depicts an embodiment of monitoring ischemic conditioning induced by a balloon situated internally within a blood vessel. FIG. 18B depicts an embodiment of a guiding catheter sensory mechanism as used for ischemic conditioning. FIG. 18C depicts another embodiment of a guiding catheter sensory mechanism as used for ischemic conditioning. FIGS. 18D and 18E depict embodiments of external sensory mechanisms as used for ischemic conditioning.

FIG. 19A depicts illustrations of expected modulations to reperfusion flow rate that can be provided. FIG. 19B depicts illustrations of expected effects on metabolic markers within a target tissue that can be expected by modulating reperfusion flow rates.

FIGS. 20A-D show data indicating variations in tissue oxygenation between individuals.

FIGS. 21A-B show analysis of data indicating variations in tissue oxygenation between individuals as a consequence of partial vessel occlusion.

### DETAILED DESCRIPTION

Without limiting the scope of the invention, it is described in connection with induction of ischemic conditioning of tissues by control occlusions. In medicine, blocking, or occluding, a blood vessel to reduce or eliminate blood flow to an area of the body has become an accepted treatment option for a wide range of circulatory and internal organ diseases. Vascular occlusion can be used for a number of reasons including: to reduce pressure on malformed (fistular), weakened (aneurismal), or leaking blood vessels; to reduce blood supply to benign or malignant tumors or growths in the body; to reduce blood supply (and therefore the overall size) of an organ or area of the body prior to other therapies or procedures, and to reroute blood supply to a different blood vessel or part of the body.

The present inventors have adapted the experimental phenomena of ischemic conditioning to useful preventative and therapeutic measures for a myriad of indications. In certain embodiments, the process is monitored and controlled as well as individualized to the unique physiology of the individual patient.

The controlled induced ischemia provides conditioning to increase effects of therapies and decrease the incidence and extent of tissue injury by several mechanisms, e.g. increased scavenging of free radicals induced by trauma and reduction in inflammation. In other embodiments, the administration of controlled induced ischemia is adapted to increase functional capillary density in desired sites with an outcome of hastened wound healing.

In an embodiment, instruments are provided to deliver atraumatic extravascular occlusions. For example, an occluding member is adjusted to reach difficult areas and is then secured around or at least partial enclosing a blood vessel with minimal maneuvering and deployment trauma. Further, in an embodiment, the occluding member or “occluder” is designed for accurate and precise applications of pressure that are repeatable and/or adjustable to maximize control of the extent, duration, and frequency of occlusions. In an embodiment, the instrument is of a profile and size that is suitable for any invasive or minimally invasive procedure. In an embodiment, monitoring of tissue ischemia and effects of ischemic conditioning in a targeted tissue is provided as feedback to further enhance the ischemic conditioning procedure.

Ischemic conditioning procedures such as preconditioning, postconditioning, and remote conditioning of blood vessels rely heavily on at least partial vascular occlusion. Brief periods of ischemia (a local shortage of oxygen-carrying blood supply) in biological tissues render that tissue more resistant to subsequent ischemic insults. Ischemic conditioning appears to be a ubiquitous endogenous protective mechanism at the cellular level that has been observed in the heart of humans and every animal species tested. This protection has also been seen in organs such as the stomach, liver, kidney, gut, skeletal tissue, urinary bladder and brain. See D M Yellon and J M Downey, “Preconditioning the myocardium: from cellular physiology to clinical cardiology,” Physiol Rev 83 (2003) 1113-1151. Furthermore, for an organ or tissue already undergoing total or subtotal ischemia, it is known that modification of blood flow conditions during the onset of resumed blood flow is able to significantly reduce reperfusion injury. Since this method begins at the onset of resuming blood flow after ischemia, it is known as postconditioning.

Non-pharmacological approaches to ischemic conditioning have included utilizing physical exercise to increase demand for oxygen and intervening ischemic occlusion procedures that are expensive and dangerous, such as angioplasty. See, inter alia, U.S. Pat. No. 6,702,820 and U.S. Patent Publication No. 2004/0255956. These planned interventions measure the effects of ischemia by certain markers in peripheral blood. However, these markers do not provide real-time contemporaneous feedback such that the conditions of ischemia can be optimized to suit the highly sensitive needs of ischemic preconditioning and postconditioning in individual patients. Accordingly, what are needed are non-pharmacologic methods and apparatus for controlling ischemic preconditioning and postconditioning based on monitoring of markers of ischemia.

Devices to induce extravascular occlusions, or occlusions from the outside of vessels, are typically clips or clamps designed in shapes with vessel contacting surfaces on their inner aspects, which surround the vessel. When occluding pressure is applied to the vessel, the vessel is compressed by a constricting force applied around its circumference. Significant trauma to the vessel often results from the use of such occluding devices. The cause of this trauma is at least partially attributed to the design of these devices and the way in which they apply compressive forces to the vessel. Specifically, the placement, extent, duration, frequency, and/or release of existing compressive forces applied to the vessel preclude adjustments to minimize trauma to the vessel, thus forcing the vessel tissue to compress uncontrollably with relatively little compliance. The compressive forces required to effect this compression and occlusion of the lumen of the vessel often result in an actual crushing of the vessel tissue, with consequent damage.

The occluding devices provided herein are adapted for ease and rapidity of loading, maneuvering, deploying, and releasing. For example, a problem is posed particularly in the settings of active bleeding and ischemic conditioning where occlusions may need to be applied in rapid sequence. Also, certain areas of the body are difficult to access and deployment of these devices is therefore often blind. Further, existing devices that might be employed often do not hold and need greater strength to manipulate anatomy. The size of a vessel to be occluded is typically limited by the size of existing devices. Additionally, timing the release of these existing devices is often uncontrolled so the duration of occlusions can be indeterminable absent a manual release of the device. Accordingly, what are needed are methods and apparatus to enable extravascular occlusions that minimize trauma and improve ease of use and clinical utility of existing devices, especially with regard to ischemic conditioning.

As used herein the term “ischemia” means lowering of baseline blood flow to a tissue. The term “hypoxia” means lowering of arterial PO₂. Both ischemia and hypoxia can be induced in tissues by partial or complete occlusion of blood supply upstream of the tissue.

By “distal extremity” it is meant the hands and feet, including the digits of the hands and feet. By “regional or local” it is meant administration to a defined area of the body as contrasted with systemic administration. In an embodiment the occlusion is sufficient to induce reactive hyperemia in the at least one limb or portion thereof.

“Reactive hyperemia” is a term that can be defined as an increase in blood flow to an area that occurs following a brief period of ischemia (e.g., arterial occlusion). One embodiment of the present invention employs controlled administrations of ischemia to condition tissues of target areas. By “target areas” it is meant areas known to exhibit injury expected to tissues during medical, surgical and other pharmacological interventions or non-pharmacological injuries. The term “ischemic conditioning” means inducing one or more episodes of ischemia that are controlled.

As used herein, the term “occlusion” means a partial or total shutting off or obstruction, usually in reference to a lumen, including a blood vessel. By “blood vessel” it is meant arteries, veins, capillaries, and any part of the cardiovascular system that functions to transport blood throughout the body. Accordingly, by “extravascular occlusion” it is meant an occlusion induced from outside a blood vessel. The term “lumen” refers to an inner space, lining, or cavity of any anatomical structure, including for example the central space in blood vessels and lymphatic vessels, the interior of the gastrointestinal tract, the pathways of the bronchi in the lungs, the interior of urinary collecting ducts, or the single pathway of the vagina. Accordingly, a “luminal tissue” is a tissue that includes the above referenced lumen and a “extraluminal occlusion” refers to an occlusion induced from outside the lumen to be occluded. Applications for occlusion of blood vessels as described herein can also be applicable to lumens that are not blood vessels. Similarly, an extraluminal occlusion by the invention as described herein can be provided to result in vascular occlusion of vessels associated with the lumen. For example, sites for extraluminal occlusions can include but are not limited to: the gastrointestinal tract, lymphatics, urinary collecting ducts, vagina, and bronchi.

As used herein the phrase “compounds that increase the bioavailability of nitric oxide (NO)” include NO precursors, NO donors and NO agonists. An example of a NO precursor is the essential amino acid substrate L-arginine from which NO is synthesized by the action of nitric oxide synthase (NOS). NO donors, which generate NO via NOS independent processes, include both fast and slow release compounds that typically release NO by either oxidation or reduction. Certain of the NO donor compounds such as nitroglycerin (an organic nitrate), which is enzymatically degraded to generate NO, have been utilized for over a century. Examples of NO donors (sometimes alternatively referred to in art as NO agonists) include the organic nitrates (e.g. glyceryl trinitrate, isosorbide dinitrate), sodium nitroprusside (SNP), syndnonimines (e.g. molsidomine, SIN-1), S-nitrosothiols (e.g. s-nitrosoglutathione), NONOates (e.g. Spermine-NONOate, DETA-NONOate), and hybrid donors such as the nitroaspirins and nicrorandil. Certain other compounds that are considered herein to fall within the definition of compounds that increase the bioavailability of NO are compounds, and metabolites thereof, that include nitric oxide chemical structures and are considered to be NO agonists such as for example minoxidil (3-hydroxy-2-imino-6-(1-piperidyl)pyrimidin-4-amine). Such compounds are considered herein to be NO agonists if their action is the same as NO, such as for example, in opening of membrane potassium channels.

Ischemic Preconditioning: The benefits of ischemic preconditioning have been observed in myocardial tissue of dogs that were pretreated by alternately manually clamping and unclamping coronary arteries to intermittently turn off the blood flow to the heart. Dogs who were treated with an optimal number of four cycles of five-minute coronary occlusion followed by five-minute reperfusion, exhibited 75% smaller infarct sizes resulting from a subsequent forty-minute coronary occlusion. Fewer than four cycles of coronary occlusion resulted in insufficient preconditioning in the dog model. Myocardial tolerance to injury also develops in response to treatment that does not include coronary occlusion (i.e., ischemia) but otherwise increases demand for oxygenated blood. In dogs, a treatment comprising of five five-minute periods of tachycardia alternating with five minutes of recovery has also been shown to reduce infarct sizes.

The myocardial resistance to infarct resulting from brief periods of ischemia has been described in other animal species including rabbit, rat and pig. Ischemic preconditioning has also been demonstrated in humans. A second coronary occlusion during the course of coronary angioplasty often results in less myocardial damage than the first. Naturally occurring ischemic preconditioning of the myocardium has been found in humans suffering from bouts of angina.

Ischemic preconditioning occurs not only in myocardial tissue but also occurs in non-cardiac tissue including kidney, brain, skeletal-muscle, lung, liver and skeletal tissue. Further, resistance to infarct exists even in virgin tissue following brief ischemia in spatially remote cardiac or non-cardiac tissue. Ischemic preconditioning also exhibits a temporal reach: an early phase develops immediately within minutes of the preconditioning ischemic injury and lasts for a few hours, and a late phase develops with apparent circadian regularity twenty four hours later and reappears cyclically over several days.

Postconditioning: Timely reperfusion to reduce the duration of ischemia is the definitive treatment to prevent cellular injury and necrosis in an ischemic organ or tissue. However, defined as reperfusion injury, damage can occur to an organ by the resumption of blood flow after an episode of ischemia. This damage is distinct from the injury resulting from the ischemia per se. One hallmark of reperfusion injury is that it may be attenuated by interventions initiated before or during the reperfusion. Reperfusion injury results from several complex and interdependent mechanisms that involve the production of reactive oxygen species, endothelial cell dysfunction, microvascular injury, alterations in intracellular Ca2+ handling, changes in myocardial metabolism, and activation of neutrophils, platelets, cytokines and the complement system. Deleterious consequences associated with reperfusion include a spectrum of reperfusion-associated pathologies that are collectively called reperfusion injury. Reperfusion injury can extend not only acutely, but also over several days following a medical or surgical intervention.

For example, even with successful treatment of occluded vessels, a significant risk of additional tissue injury after reperfusion may still occur. Typically, reperfusion after a short episode of myocardial ischemia is followed by the rapid restoration of cellular metabolism and function. However, if the ischemic episode has been of sufficient severity and/or duration to cause significant changes in the metabolism and the structural integrity of tissue, reperfusion may paradoxically result in a worsening of function, out of proportion to the amount of dysfunction expected simply as a result of the duration of blocked flow. Although the beneficial effects of early reperfusion of ischemic myocardium with thrombolytic therapy, PTCA, or CABG are now well established, an increasing body of evidence indicates that reperfusion also induces an additional injury to ischemic heart muscle, such as the extension of myocardial necrosis, i.e., extended infarct size and impaired contractile function and metabolism. Hearts undergoing reperfusion after transplantation also undergo similar reperfusion injury events. Similar mechanisms of injury are observed in all organs and tissues that are subjected to ischemia and reperfusion.

Thus, in general, all organs undergoing reperfusion are vulnerable to reperfusion injury. Postconditioning is a method of treatment for significantly reducing reperfusion injury to an organ or tissue already undergoing total or subtotal ischemia. Postconditioning involves a series of brief, iterative interruptions in arterial reperfusion applied at the immediate onset of reperfusion. The bursts of reflow and subsequent occlusive interruptions last for a matter of seconds, ranging from 60 second intervals in larger animal models to 5-10 second intervals in smaller rodent models. Preliminary studies in humans used 1 minute intervals of reperfusion and subsequent interruptions in blood flow during catheter-based percutaneous coronary intervention (PCI).

The spatial and temporal characteristics of ischemic preconditioning and postconditioning may be a manifestation of complex interactions between various underlying phenomena. The numerous biochemical and cellular mechanisms underlying the phenomena of ischemic conditioning are still being researched and are not fully understood. These research efforts have been motivated at least in part by the hope of developing pharmaceutical drugs which would provide the infarct sparing effect of ischemic conditioning.

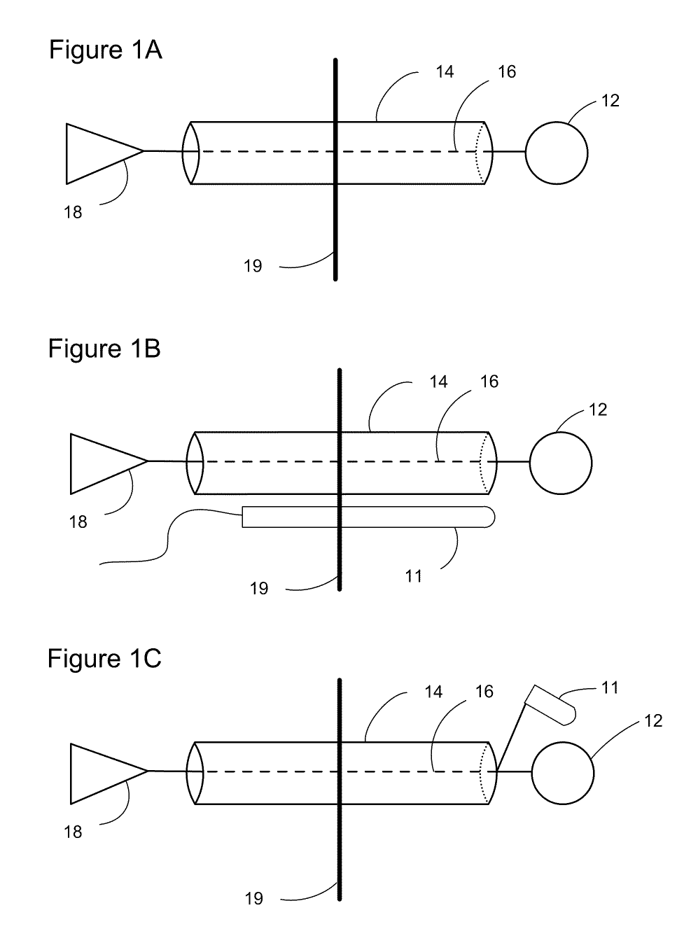

System for Extravascular Occlusion of a Blood Vessel: In an embodiment, a system for controlling extravascular occlusions is provided. As depicted in FIGS. 1A-C, an embodiment of a system for controlling extravascular occlusions includes an occluding member (12), a hollow member (14), an occluding mechanism (16), and a control mechanism or controller (18). The hollow member can be guided through an incision from the exterior to the interior of a body, the boundary of which is depicted by a line (19). From the interior of the body, the hollow member can be a tubing of any suitable length, material, and thickness that can be guided to a blood vessel for deployment of the occluding member. The occluding mechanism can be provided to deploy, position, and tighten the occluding member against a blood vessel to induce at least a partial occlusion. The control mechanism or controller can allow for control of the occluding mechanism to guide placement, amount, duration, frequency, and release of the occlusion.

In an embodiment, the system for controlling extravascular occlusion can be adapted for any suitable configuration for surgery. In an embodiment, the entire system for extravascular occlusion can be minimally invasive and/or atraumatic. For example, in an embodiment, all the components of the system are configured in suitable endoscopic or laparoscopic arrangements. Embodiments of suitable endoscopic configurations as adapted for systems for extravascular occlusion are depicted in FIGS. 1B and 1C. As depicted, an endoscope (11) fitted with a camera and/or light can be guided into the body through a separate incision site or through the tubing to provide imaging of the system.

Similarly, the system for controlling extravascular occlusion can be adapted to include and/or fit other suitable surgical instruments, including but not limited to: graspers, especially tweezers and forceps; known clamps and occluders for blood vessels and other organs; retractors, used to spread open skin, ribs and other tissue; distractors, positioners and stereotactic devices; mechanical cutters (scalpels, lancets, drill bits, rasps, trocars, etc.); dilators and specula, for access to narrow passages or incisions; suction tips and tubes, for removal of bodily fluids; irrigation and injection needles, tips and tubes, for introducing fluid; tyndallers, to help “wedge” open damaged tissues; powered devices, such as drills, dermatomes; scopes and probes, including fiber optic endoscopes and tactile probes; carriers and appliers for optical, electronic and mechanical devices; ultrasound tissue disruptors, cryotomes and cutting laser guides; and/or measurement devices, such as rulers and calipers.

Further, in an embodiment, components of the system such as the hollow member, occluding mechanism, and control mechanism can be any that are well known in the art and suitable for the invention as described herein. For example, the system can include any of numerous tactile feedback triggered handle and gun delivery systems that are currently available for surgical procedures. In an embodiment, one or more of the steps of the method as described herein can be simultaneous, continuous, randomized, or combinations thereof. In an embodiment, the occluding members can be manual, automated, motorized, and/or programmable by a device in the control mechanism. A device for controlling the timing and amount of occlusion and release of the blood vessel can be inside or outside of the body.

In an embodiment, all or a portion of the instrument as described herein is adapted for disposable use. In other embodiments, all or a portion of the instrument as described herein is designed to be cleaned, sterilized and reused. All or a portion of the instrument can be designed for short term or temporary placement and use, or may be permanently or semi-permanently deployed. In an embodiment, the control mechanism is adapted for constriction of the occluding member, expanding the occluding member, or combinations thereof. For example, a motorized mechanism can allow for both constriction and expansion of an occluding member. Constriction and expansion can also be facilitated by a biological response. For example, the occluding member can be composed of a material that expands upon exposure to defined thresholds of water, temperature, or combinations thereof.

Occluding Members: Considering the occluding members in more detail, extravascular occlusions in an embodiment can be provided by one or more pistons, jaws, coils, steerable clamps, inflation, timed release, or combinations thereof. Such occluding members can provide minimal trauma to the blood vessel and/or surrounding areas. In an embodiment, one or more of the edges of the instrument can have dulled edges to minimize unnecessary damage to contacting tissues and blood vessels. In an embodiment, the occluding members can be shaped, e.g. grooved or angled, to minimize trauma to the blood vessel. In an embodiment, one or more jaws can be composed of a material that is capable of cushioning the blood vessel to minimize trauma. For example, suitable polymers are well known that can be adapted to suit the invention as described herein.

In an embodiment, an occluding member improves ease of mounting the instrument on a blood vessel. In an embodiment, an occluding member can be designed to secure the blood vessel within its interior. In an embodiment, an occluding member can be provided with a stabilizing mechanism to assure mounting and securing of the occluding member around the blood vessel. In an embodiment, a single compression by an occluding member can compress more than one blood vessel at once. In an embodiment, several occluding members can be used simultaneously, sequentially, and/or at random.

In an embodiment, an occluding member can be manual, semi-automated, automated, and/or motorized. In an embodiment, an occluding member can have a base state of occluding the vessel and can be adjusted to release the occlusion, and/or vice versa where the base state is open and an adjustment can occlude the vessel. In an embodiment, motions such as opening, closing, loosening, tightening, or combinations thereof, of the occluding members can be controlled by a device connected to the occluding member. The method of actuating the compression and decompression can include use of mechanical pressure like air compression and/or electromagnetic force. Accordingly, the compression can open and close the occlusion when needed with or without manual handling.

In an embodiment, a programmable device that controls compression and decompression of the occluding members can be external of the body, internal of the body, and/or combinations thereof. In an embodiment, the occluding member can be fitted with sensors (e.g. palpation, pressure, blood flow, Doppler, temperature, and/or ischemia monitoring sensors) which are able to accurately determine the pressure that is being applied to the vessel and/or the ischemia induced by the occlusion. In an embodiment, motorizing and/or automating the occluding member provides the significant advantage of precise control of repeated occlusions. Further, manual controls can also be provided to ensure stabilization and/or safety releases of the vessel.

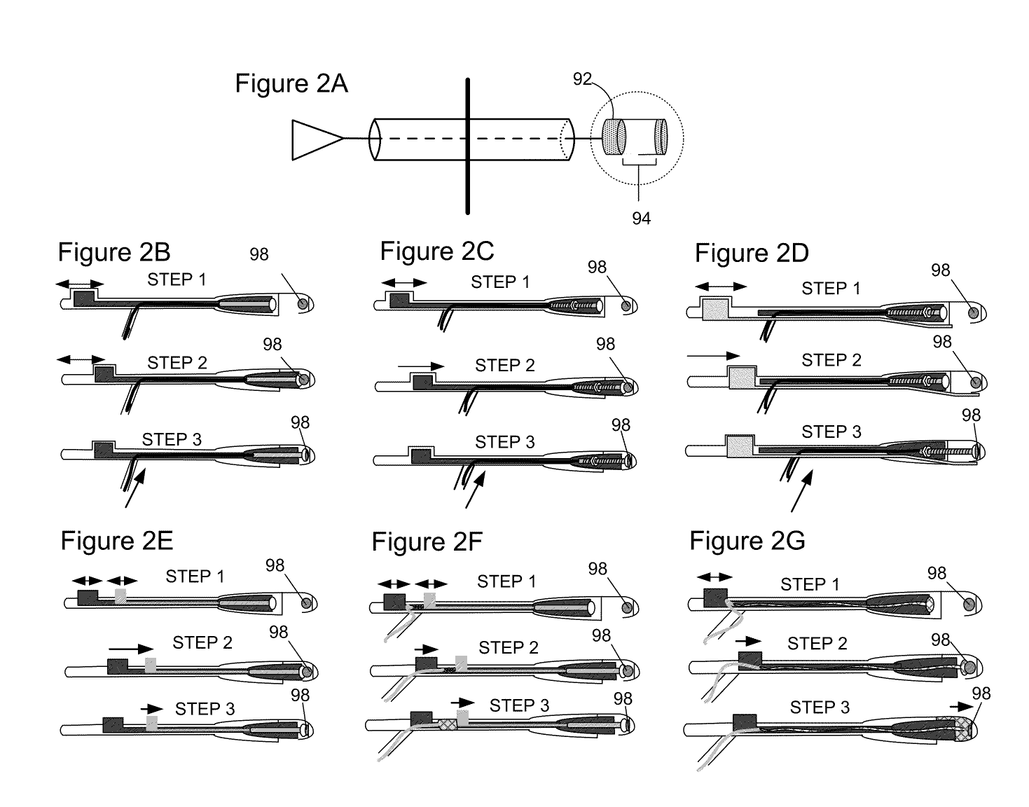

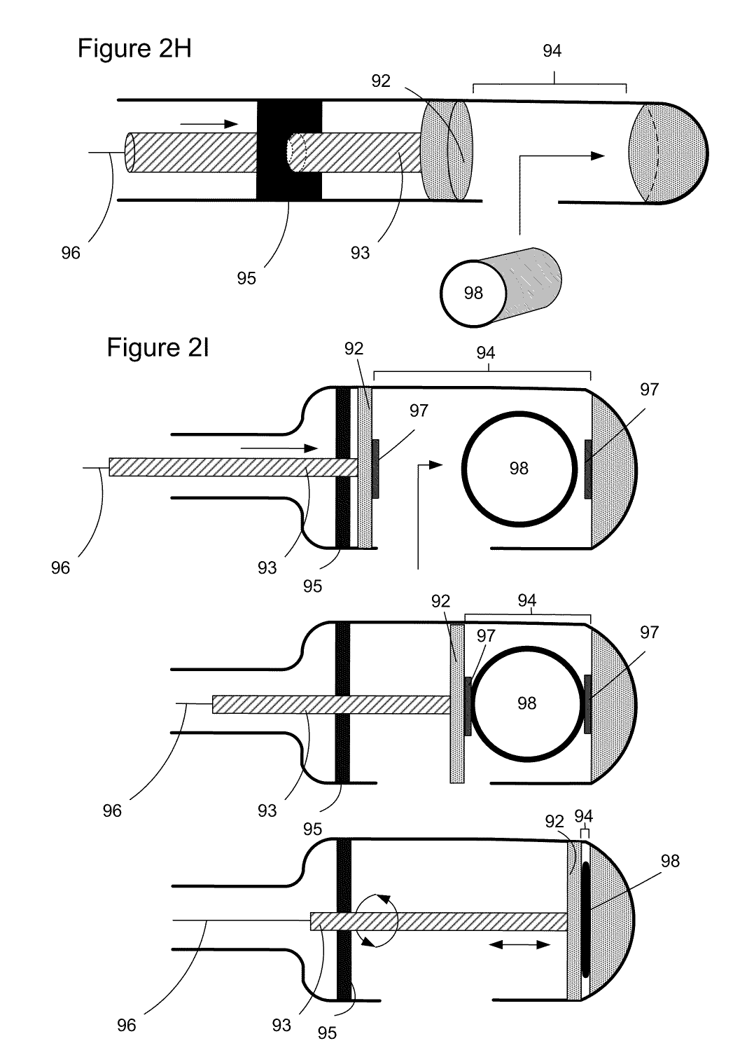

FIGS. 2A-I depict embodiments of pistoned occluding members capable of extravascular occlusion. In an embodiment, as depicted in FIG. 2A, the instrument has an occluding member with a piston (92) and at least one interior space (94) within the piston (92). FIGS. 2B-2G depict embodiments of cross-sectional views of a semi-automated and/or manual instrument that can be delivered so that a blood vessel (98), also shown in cross-sectional view, is captured within in an interior space within the instrument. In an embodiment, the piston is delivered so that the blood vessel is inside a cylinder attached to the piston, as indicated by Step 1. For example, the tip of the piston can be curved into a shape as depicted to allow for hooking around a blood vessel. The blood vessel can then be secured inside the interior space of the piston by manual mechanisms as depicted by Step 2. Step 3 shows the compression of the blood vessel by various automated and manual mechanisms.

FIG. 2B depicts a semi-automated embodiment with manual securing by a switch slide of the piston in Step 2. The blood vessel is enclosed by moving the piston forward enough into the interior space of the hook shaped curve of the enclosure to prevent the blood vessel from slipping out. In an embodiment, the hooked enclosure can move back towards the piston to enclose the vessel. Step 3 is depicted as an automated sliding compression and decompression of the blood vessel by the piston that is controlled by electric signals coming from a wire which, in turn, is connected to a controller. FIG. 2C also depicts a semi-automated embodiment with manual securing by a switch slide of the piston in Step 2. Step 3 however is depicted as an automated rotationally driven compression and decompression of the blood vessel that is controlled by electric signals coming from the wire. This design includes two concentric rigid plastic or similar housings. Both are cylindrical and hollow. The inner enclosure houses an incompressible catheter clamp guidewire. Suitable incompressible yet flexible guidewires that allow for flexible strain relief are well known in the art. The inner enclosure is attached to a slide on the outer enclosure. This mechanism allows the inner enclosure to be advanced and retracted independently of the outer enclosure. A stationary nut is affixed inside of the tip of the occluding member. A rotor is threaded into the stationary nut. Once the vessel of interest is positioned inside of the interior space of the piston, the inner housing is advanced to immobilize the vessel in the clamp channel so that it does not slip out. The motor assembly can be activated to rotate a guide wire and advance the piston into the piston channel to ultimately apply pressure against the vessel and the piston wall. In an embodiment, by taking input from a pressure feedback, the motor assembly can stop the advancement of the clamp face when a predetermined pressure has been reached.

FIG. 2D again depicts a semi-automated embodiment with manual securing by a switch slide in Step 2, but instead of the slide moving the piston, a securing member is provided to slide and secure the outside of the cylinder to prevent the blood vessel from slipping out. Step 3 is depicted as an automated rotationally driven compression and decompression of the blood vessel that is controlled by electric signals coming from a wire. FIG. 2E depicts a fully manual embodiment with manual securing of the blood vessel by a switch slide of the piston in Step 2 and also manual compression and decompression of the blood vessel by a separate switch slide in Step 3. FIGS. 2F and 2G show embodiments of manually securing the blood vessel by a switched slide of the piston in Step 2, but precisely controlled compression and decompression of the blood vessel is then achieved by inflation and deflation. Inflation can be particularly advantageous to accurately measure the amount of pressure being put into the sensitive procedure. FIG. 2F depicts actuation of a sliding piston by inflation in Step 3 while FIG. 2G depicts direct inflation of the piston to achieve occlusion in Step 3.

The cylinder of the piston can be open or closed prior to delivery. Such a pre-opened design can improve access and delivery of the instrument as positioning of the piston around the blood vessel only requires a hooking motion of the piston, as depicted in the cross-sectional views of the rotationally driven piston embodiments in FIGS. 2H-I. In an embodiment, the piston can perform the securing of Step 2 automatically upon delivery to the blood vessel. For example, a sensor can be adapted to deploy a securing mechanism around a blood vessel upon contact with the blood vessel. Further, in an embodiment, the securing mechanism around the blood vessel can be an automated and/or motorized process where the user can make a simple motion, e.g. pushing a button or pulling a trigger, which deploys a securing mechanism. In an embodiment, delivery of the pistoned instrument can be further enhanced by modifying the edges to minimize trauma to the blood vessel. For example, a dulled weight can be placed on an end of the instrument to aid in softening contact with the blood vessel and/or minimize any slipping of the blood vessel within the interior space. Such dulled weights are known in the art particularly in surgical technologies and can be any suitable for the invention as described herein.

In an embodiment, at least a partial occlusion can result from compression of the blood vessel by reducing the interior space. Any suitable mechanism for reducing the interior space can be used. For example, FIGS. 2H-I depict cross sectional views of embodiments of a piston that can be rotationally driven to compress and decompress a blood vessel secured within the cylinder attached to the piston. Driving all or a portion of the piston (92) can reduce the interior space (94) to compress the blood vessel (98). A rotatonally driven mechanism that is able to tighten, lock, and release the piston (92) can also facilitate at least a partial occlusion of a blood vessel (98) by reducing an interior space (94). A piston can be freewheeling and mounted on a threaded slide (93) that allows it to move back and forth. The threaded slide (93) can be fed through a coupling nut (95) that is attached to the enclosure. As the slide rotates, the stationary coupling nut will advance or retract the entire piston assembly. An attachment mechanism (96) is fixed to the end of the slide that connects directly to a motor or other engine or driving force. Sensors (97) for monitoring of parameters, including but not limited pressure feedback, temperature, ischemia, and/or blood flow; can also be provided. Further, in an embodiment, the same mechanism that facilitated the reduction in interior space can allow for release of all or part of the occlusion by increasing the interior space. In an embodiment, such a design can allow for motorized and precise control of actuation and/or compression and decompression of the blood vessel by the pistoned occluding member.

In an embodiment, the piston movement back and forth within the cylinder can be automated by any suitable mechanism that is known in the art. For example, the freewheeling piston can be moved by a threaded slide that can be rotated by motorized wire and/or propellers. In an embodiment, threading of the slide and rotational force is not necessary to move the piston. For example, a slide without threading that is actuated by wire, propellers, compressed air, and/or air inflation and deflation of an attached bladder can be provided. In an embodiment, movement of the slide back and forth can be accomplished by any mechanical means suitable in the art for allowing precise control of the amount of compression and decompression that is applied to the blood vessel.

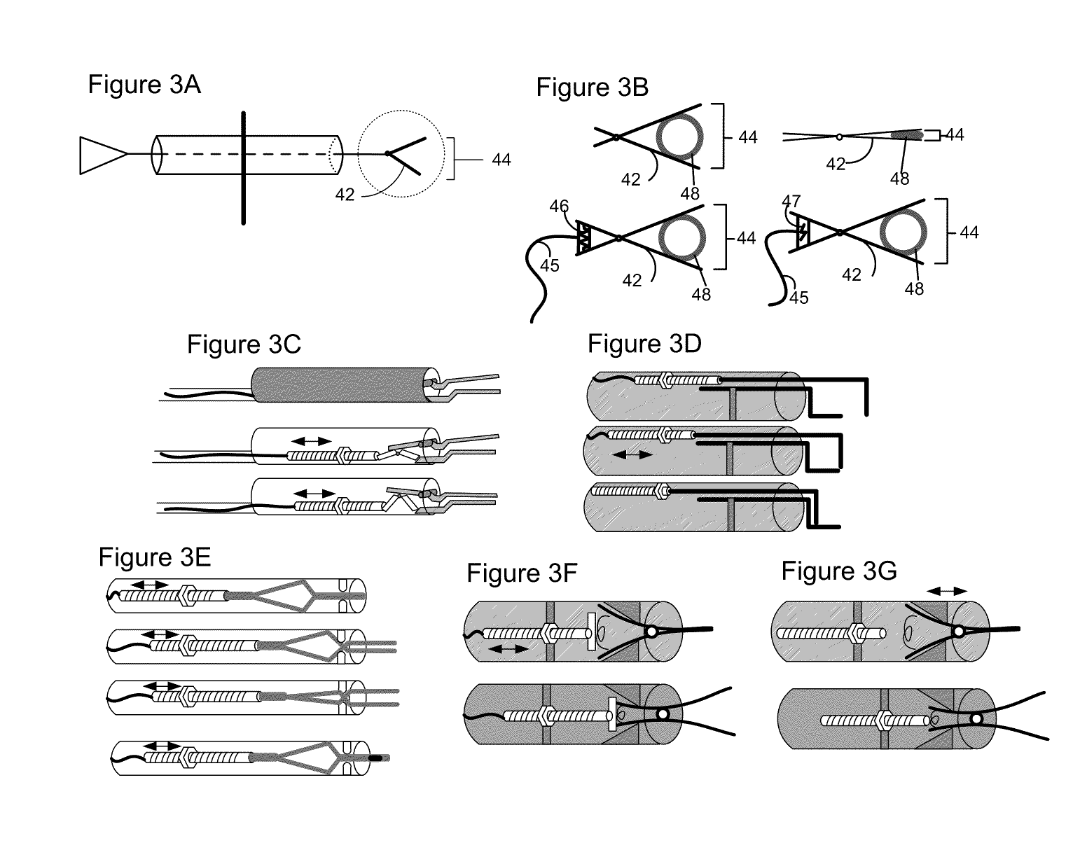

FIGS. 3A-D depict embodiments of jawed occluding members capable of extravascular occlusion. Similar to the pistoned member with an interior space as described herein, in an embodiment, one or more sets of jaws (42) contains at least one interjaw space (44) between two individual jaws. The instrument can be delivered so that a blood vessel (48) is in the interjaw space. In an embodiment, at least a partial occlusion results from compression of the blood vessel by reducing the interjaw space. Any suitable mechanism for reducing the interjaw space can be used. For example, closing all or a portion of the jaws can reduce the interjaw space to compress the blood vessel. Similarly, a spring compression mechanism that is able to tighten, lock, and release the jaws can also facilitate at least a partial occlusion of a blood vessel by reducing an interjaw space. Further, in an embodiment, the same mechanism that facilitated the reduction in interjaw space allows for release of all or part of the occlusion by increasing the interjaw space.

In an embodiment, the jawed occluding member is advantageously automated for occlusion and release of the occlusion. For example, jawed occluding members that are well known in the art, e.g. atraumatic “bulldog” clamps, can be programmed by a device connected to the clamps to occlude and release according to a schedule. Motions such as opening, closing, loosening, tightening, or combinations thereof, of the clamps are controlled by a device connected to the clamp via one or more electrical communication lines (45). The method of actuating the clamp can include use of mechanical pressure like air or spring compression (46), electromagnetic force (47), and/or rotational force (49). Accordingly, the clamp can open and close when needed with or without manual handling. In an embodiment, the clamp can be actuated at the jawed end of the clamp instead of, or in addition to, the controlling end. In an embodiment, the programmable device that controls occlusion and release of the jawed occluding members can be external of the body, internal of the body, and/or combinations thereof.

In an embodiment, the jawed occluding members can be precisely controlled and/or shaped to minimize trauma to the blood vessel. For example, as depicted in FIG. 3C, the jaws can be precisely controlled by rotational force, as described herein, and configured by a hinge which allows parallel, flat, and atraumatic compression and decompression of the blood vessel. Flat and atraumatic compression can also be achieved by control of separate perpendicular jaws as depicted in FIG. 3D. Further, as depicted in FIG. 3E, the jaws can be configured in a “tweezer” shape to allow for the jaws contacting the vessel to be in parallel and flat configuration. As the “tweezer” is precisely moved forward against pressure points of the cylinder, the jaws can open and close around the vessel. Similarly, FIG. 3F depicts controlled movement of jaws against pressure points in the enclosure to pivot open the jaws around the vessel. Alternatively, FIG. 3G depicts movement of the enclosure instead of the jaws to move the pressure points backward to open the jaws. In an embodiment, the one or more jawed occluding members can be of any length and width that is known in the art suitable for occlusions as described herein. In an embodiment, the jawed occluding members can be particularly advantageous for reaching smaller blood vessels as the size of a jawed device can be minimal. In an embodiment, one or more jaws can be composed of a ridged design or material that is capable of cushioning the blood vessel to minimize trauma. For example, suitable atraumatic microgrooves and polymers are well known that can be adapted to suit the invention as described herein.

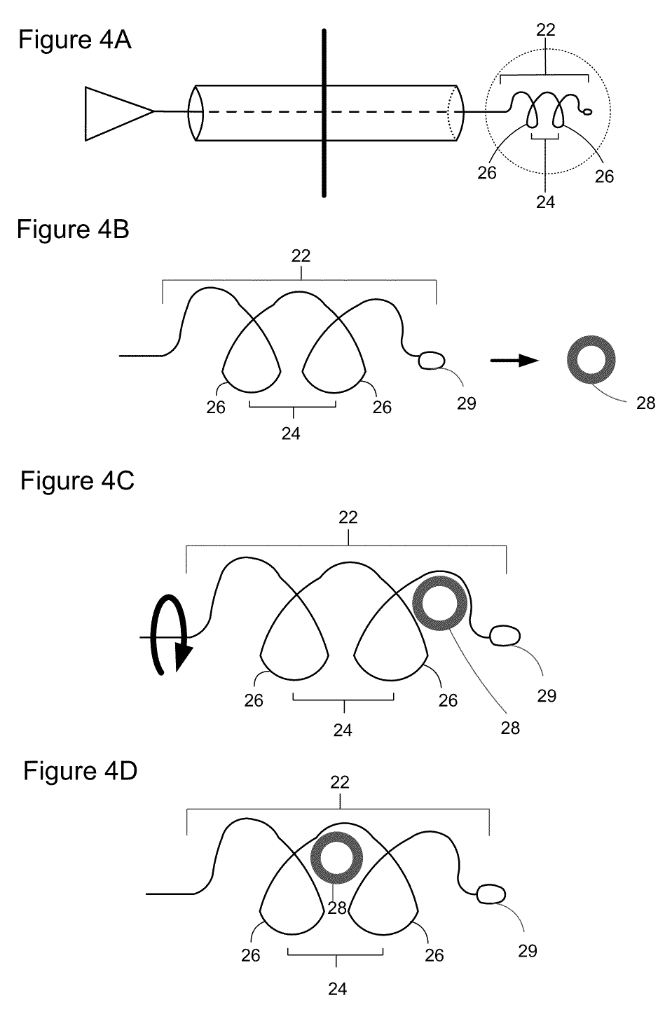

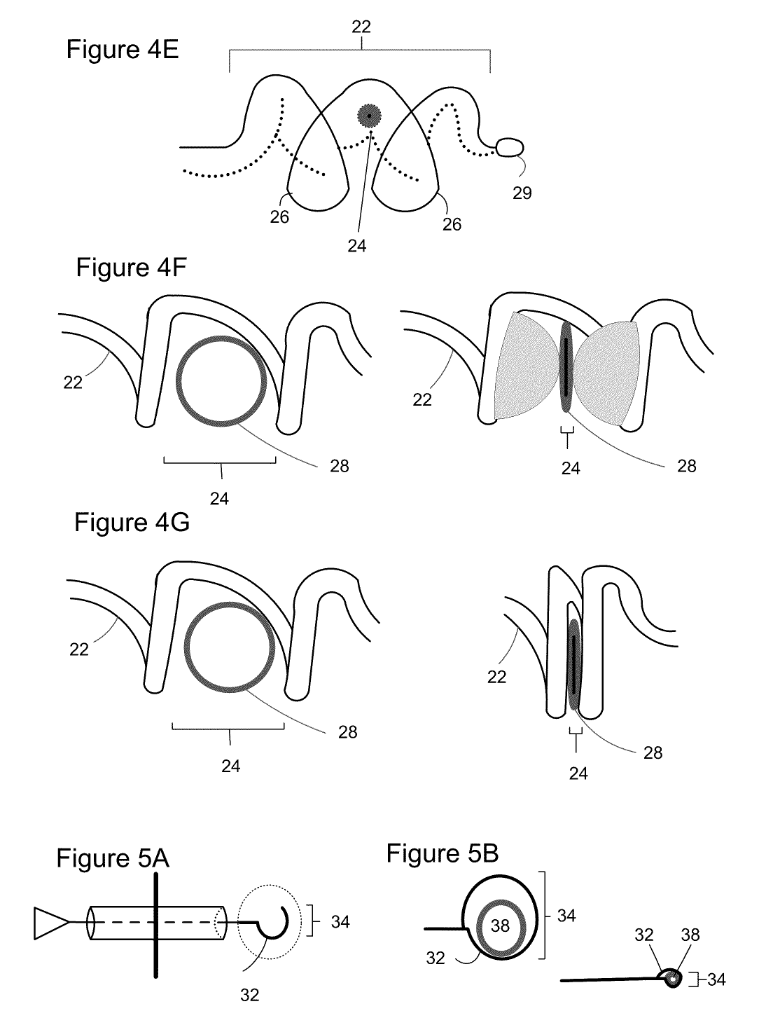

FIGS. 4A-G depict embodiments of coiled occluding members capable of extravascular occlusion. In an embodiment, as depicted in FIG. 4A, the instrument can have an occluding member with a coil (22) and can contain at least one intercoil space (24) between two individual coils (26). FIGS. 4B-4D depict embodiments of the instrument that can be delivered so that a blood vessel (28), as shown in cross-sectional view, is in the intercoil space. In an embodiment, the coil can be pre-coiled into a spiral shape prior to delivery. Such a pre-coiled design can improve access and delivery of the instrument as positioning of the coil around the blood vessel only requires a winding motion of the coil, as depicted in an embodiment in FIG. 4C. In an embodiment, the coil can be self-coiling upon delivery to the blood vessel. For example, a steerable wire can be adapted to coil around a blood vessel upon contact with the blood vessel. Further, in an embodiment, the self-coiling around the blood vessel can be an automated and/or motorized process where the user can make a simple motion, e.g. pushing a button or pulling a trigger, which instructs a steerable wire to shape into a coil. In an embodiment, delivery of the coiled instrument can be further enhanced by modifying the edges to minimize trauma to the blood vessel. For example, a dulled weight (29) can be placed on an end of the coil to aid in softening contact with the blood vessel and/or minimize any slipping of the blood vessel within the intercoil space. Such dulled weights are known in the art particularly in catheter technologies and can be any suitable for the invention as described herein.

In an embodiment, at least a partial occlusion can result from compression of the blood vessel by reducing the intercoil space. Any suitable mechanism for reducing the intercoil space can be used. For example, FIGS. 4E and 4F depict cross sectional views of embodiments of a coil that can be deflated and inflated around a blood vessel. Inflating all or a portion of the coil (22) can reduce the intercoil space (24) to compress the blood vessel (28). Similarly, FIG. 4G depicts cross sectional views of embodiments of a coil that is decompressed and compressed around a blood vessel. A spring compression mechanism that is able to tighten, lock, and release the coil (22) can also facilitate at least a partial occlusion of a blood vessel (28) by reducing an intercoil space (24). Further, in an embodiment, the same mechanism that facilitated the reduction in intercoil space can allow for release of all or part of the occlusion by increasing the intercoil space.

FIGS. 5A and 5B depict embodiments of steerable occluding members capable of extravascular occlusion. The steerable occluding member (32) can be flexible and delivered to wrap around a blood vessel (38) so that the blood vessel is in an interior space (34). In an embodiment, one end of the occluding member can be steered to wrap around a blood vessel and secured onto another portion of the same occluding member to encircle around the blood vessel. In an embodiment, at least a partial occlusion can result from compression of the blood vessel by reducing the interior space. Any suitable mechanism for reducing the interior space can be used. For example, inflating all or a portion of the steerable occluding member can reduce the interior space to compress the blood vessel. Similarly, a spring compression mechanism that is able to tighten, lock, and release the occluding member can also facilitate at least a partial occlusion of a blood vessel by reducing an interior space. Further, in an embodiment, the same mechanism that facilitated the reduction in interior space can allow for release of all or part of the occlusion by increasing the interior space.

In an embodiment, the steerable occluding mechanism can be simply delivered via a steerable wire to improve delivery and access to the blood vessel. Suitable technologies for controllable steerable wires are numerous and well known in the art. The flexibility of such wires can allow for access to blood vessels that other occluding members could not be applied to. For example, smaller arteries that require occlusion can be easier to locate and occlude via a steerable wire instead of a conventional clamp or clip.

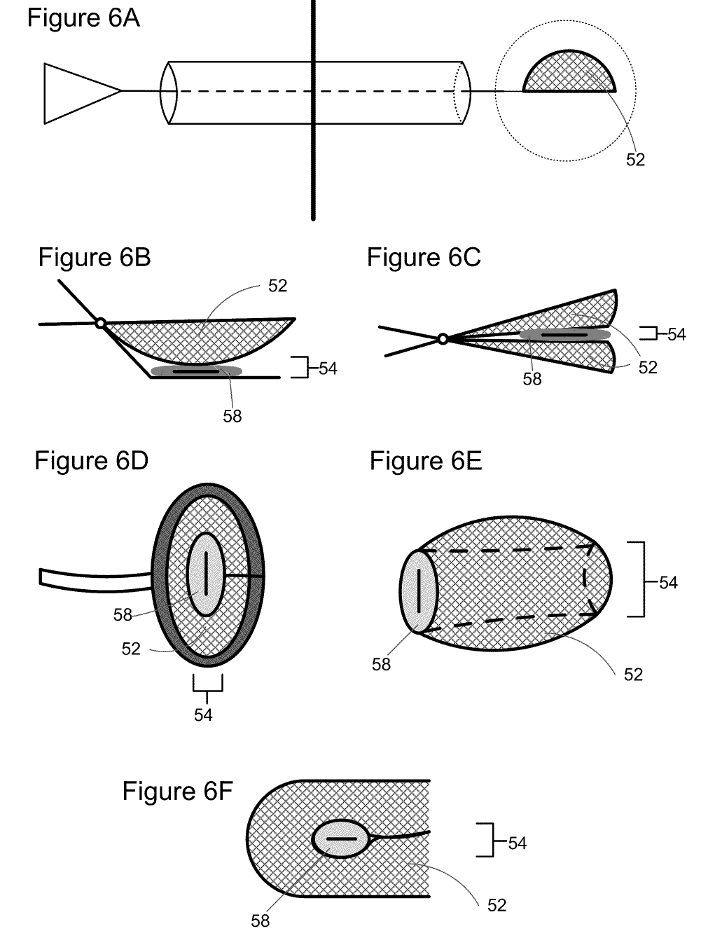

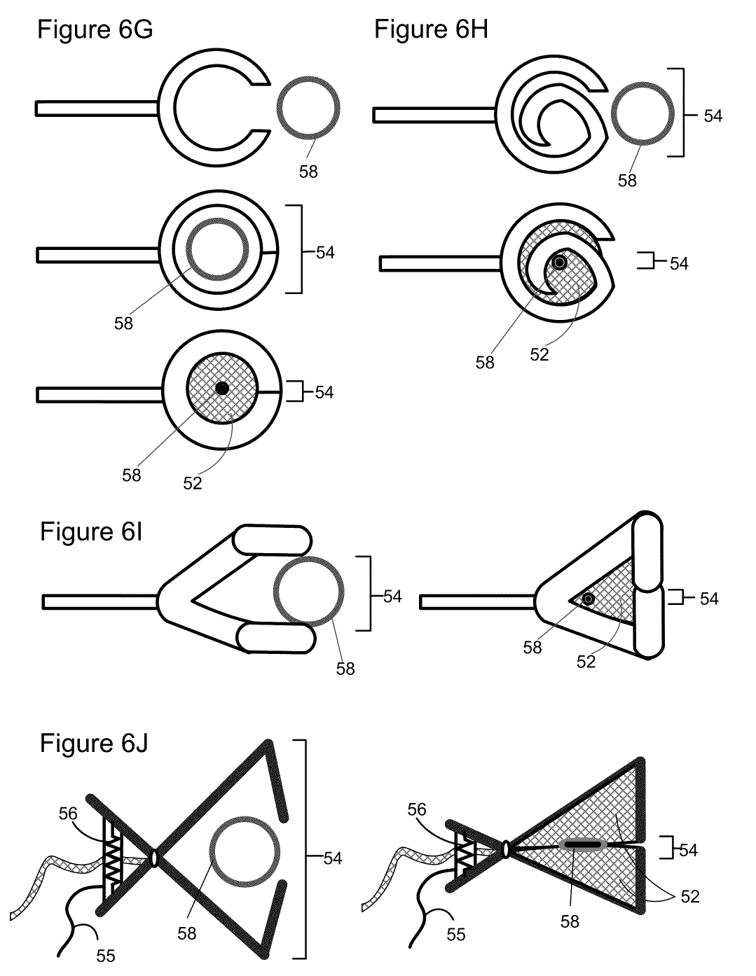

FIGS. 6A-J depict cross sectional views of embodiments of inflatable occluding members capable of extravascular occlusion. In an embodiment, a compliant balloon (52) can contain at least one interior space (54) between itself and/or another surface. Examples of suitable inflatable occluding members can include but are not limited to: a balloon disposed against an opposing hard surface such as a jaw or anatomical structure; a clamp with inflatable balloons attached; a ring shaped inflatable balloon; an extravascular tube; an extravascular suture; a locking clamp; an extravascular spiral, an extravascular shell, or combinations thereof. In an embodiment, the instrument can be delivered so that a blood vessel (58) is in the interior space. In an embodiment, at least a partial occlusion can result from compression of the blood vessel by reducing the interior space. For example, inflating all or a portion of an inflatable balloon can reduce the interior space between the balloon and an opposing surface to compress an encircled blood vessel. In an embodiment, deflation can allow for release of all or part of the occlusion by increasing the interior space.

The inflatable occluding members can be particularly advantageous not only for minimizing trauma to vessels by cushioning the contact via inflation, but also for allowing automation and/or programming of repeated occlusions and releases. Mechanisms for automating and programming inflation and deflation of various fluids are well known and any that are suitable for the invention as described herein can be used. Accordingly, accuracy of timing of occlusions and ease of use can be improved over manual methods. For example, as depicted in the embodiment of FIG. 6C, wedge shaped balloons attached to one or more hard jaws can allow for gradient compression of a blood vessel. Since the smaller portions of the wedge balloon will inflate before the larger portions, compression of a blood vessel within the interjaw space will be gently cushioned as the one or more balloons roll over the blood vessel to induce gradient compression and at least a partial occlusion. In an embodiment, a jaw can have one or balloons attached, each with one or more separate inflatable compartments to aid in inflating with gradient compression. Similarly, balloons disposed against a hard surface such as an anatomical structure and/or an opposing member of the instrument (as depicted in the embodiments of FIG. 6B and the hard shell casing of FIG. 61) can achieve compression through a reduction of the interior space between the balloon and the hard surface. In a ring shaped embodiment, such as the embodiment depicted in FIG. 6D, a ring shaped balloon can be inflated against an outer ring to compress a blood vessel upon inflation. In an embodiment, the ring shaped balloon can be adapted to compress a blood vessel upon inflation without requiring an outer ring. A tube shaped embodiment, such as the embodiment depicted in FIG. 6E, can be adapted achieve compression of the blood vessel through inflation upon itself. In a suture embodiment, such as the embodiment depicted in FIG. 6F, a suture can be wrapped around the blood vessel and inflated to compress the blood vessel. In an embodiment, one or more inflatable occluding members can secure the blood vessel prior to inflation. For example, FIG. 6G depicts an embodiment of a locking clamp that can be adapted to be closed around a blood vessel prior to inflation. Further, FIG. 6H depicts an embodiment of a spiral shaped casing that can secure a blood vessel prior to inflation.

In an embodiment, the inflatable occluding members can also be advantageously automated for occlusion and release of the occlusion. For example, FIG. 6J depicts embodiments of inflatable occluding members that can be automated for extravascular occlusion. Inflatable occluding members as described herein, such as the depicted inflatable jaws, and that are well known in the art, e.g. atraumatic vascular clamps, can be programmed by a device to occlude and release according to a schedule. Motions such as inflation, deflation, opening, closing, loosening, tightening, or combinations thereof, can be controlled by the device connected to the occluding member and/or clamp via one or more electrical communication lines (55). The method of actuating the clamp can include use of mechanical pressure like air or spring compression (56) and/or electromagnetic force. Accordingly, the clamp can open and close when needed with or without manual handling. In an embodiment, the inflatable occluding member can be composed of a biodegradable material and be implantable so that it can be delivered to the blood vessel and control multiple occlusion and releases before biodegrading. In an embodiment, such a biodegradable inflatable occluding member can be delivered with a biodegradable delivery system to allow for implanting without any resulting procedure to withdraw the occluding member and/or delivery system.

In another embodiment of the invention, apparatus for inducing ischemia in a tissue includes use of one or more releasable “minimally invasive cuffs,” small cuffs adapted to occlude blood supply to a tissue when tightened. The occlusion of blood supply can be partial or complete. In an embodiment the minimally invasive cuffs can include inflatable cuffs designed to be externally situated around at least one blood vessel. In an embodiment, the minimally invasive cuff can be delivered to be externally situated around the at least one blood vessel by a separate delivery device. The apparatus can include a pump that inflates the minimally invasive cuff and thereby occludes the blood supply according to a schedule and can further include one or more distal sensory mechanisms, drug infusion systems, heating mechanisms for intermittent heating of at least one hand or foot, or combinations thereof. The schedule can instruct tightening the cuffs in either a cyclical or sustained manner. The apparatus can be manual in operation or can be automated.

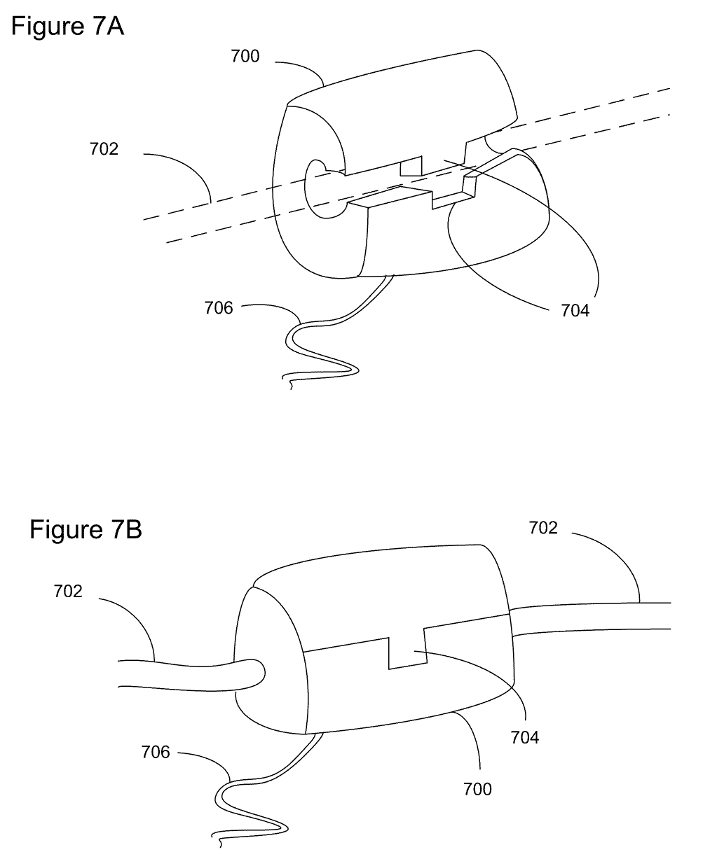

In an embodiment of the invention, ischemia is implemented by minimally invasive cuffs (700) that are secured around one or more blood vessels (702) of the patient, as depicted in FIGS. 7A & 7B. The minimally invasive cuffs can be secured around blood vessels via locking mechanisms in embodiments as depicted. The minimally invasive cuffs (700) are wrapped around a blood vessel (702), locked and unlocked by a releasable lock (704), and inflated or deflated through a fluid input (706). The minimally invasive cuffs can be locked over one or more locations of the blood vessel for compression sufficient to occlude blood flow to downstream tissue. For example, the minimally invasive cuff can be locked and compressed to partially or completely occlude blood flow. Compression of the cuff can be sustained to hold a partial or complete occlusion, and can be adjusted to allow for cyclical occlusions. To release blood supply, the minimally invasive cuff can be decompressed. The duration, frequencies, and effects of ischemia from minimally invasive cuffs to downstream tissues vary by therapeutic targets. Although only a single tooth releasable locking mechanism (704) is depicted in FIGS. 7A & 7B, any suitable releasable lock mechanisms that are well known in the art can be applied. Also, the minimally invasive cuffs, releasable locks, and fluid inputs can be any that are suitable for effecting ischemic conditioning as described herein.

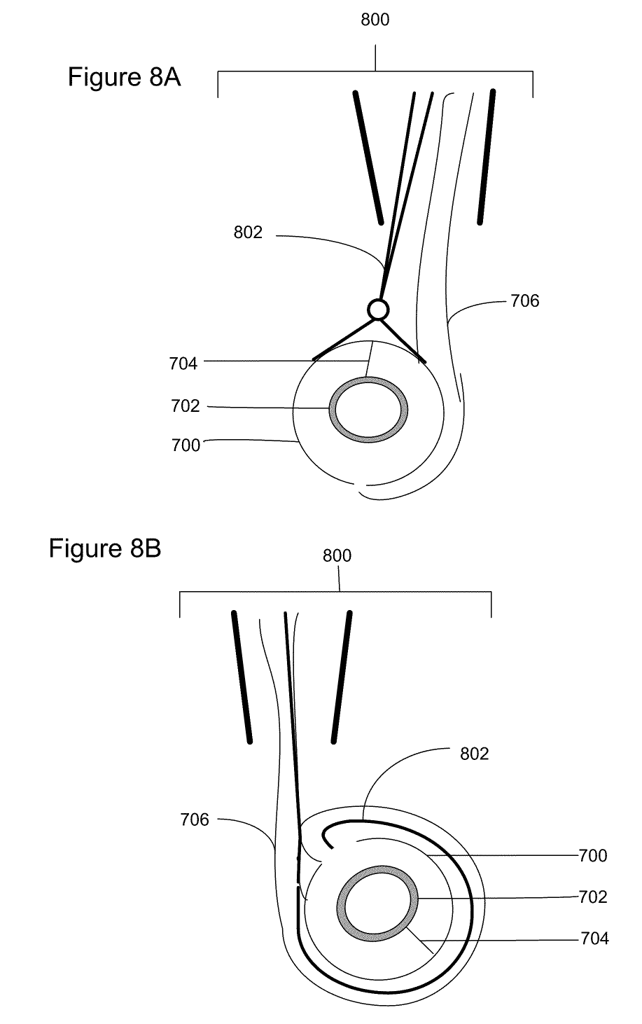

A minimally invasive cuff as delivered by minimally invasive delivery systems is embodied in FIGS. 8A & 8B. FIGS. 8A & 8B depict a cross-sectional side view perspective of cuff delivery system embodiments. The delivery systems depicted include a minimally invasive delivery device (800) that provides the cuff (700) for delivery. The delivery device (800) can situate the cuff (700) around the blood vessel (702) and lock the releasable lock (704) via inflation or a clamp (802). Once the cuff (700) is situated around the blood vessel (702), the delivery device (800) can be withdrawn and the cuff (600) can be compressed and released via a fluid input (706).

In an embodiment, inflation alone can lock the releasable lock (704). In another embodiment, the delivery device (800) can also be capable of carrying and instructing a clamp (802) for locking and releasing the cuff (800). Locking and releasing the cuff can be facilitated by any clamp suitable in the art, i.e. jawed, fixed, flexible, steering, etc. The clamp can be located internal or external of the cuff. FIG. 8A depicts a fixed jawed clamp (802) located external of the cuff with capability to lock the cuff via clamp jaws. FIG. 8B depicts a flexible or steerable clamp (802) located internal of the cuff with capability to lock the cuff by guiding the cuff to roll onto itself as depicted. Many suitable delivery devices to puncture tissues, deliver cuffs, and lock cuffs are well known in the art. The delivery device can also include any other features well known in the art suitable for deployment and retrieval of the minimally invasive cuffs as described.

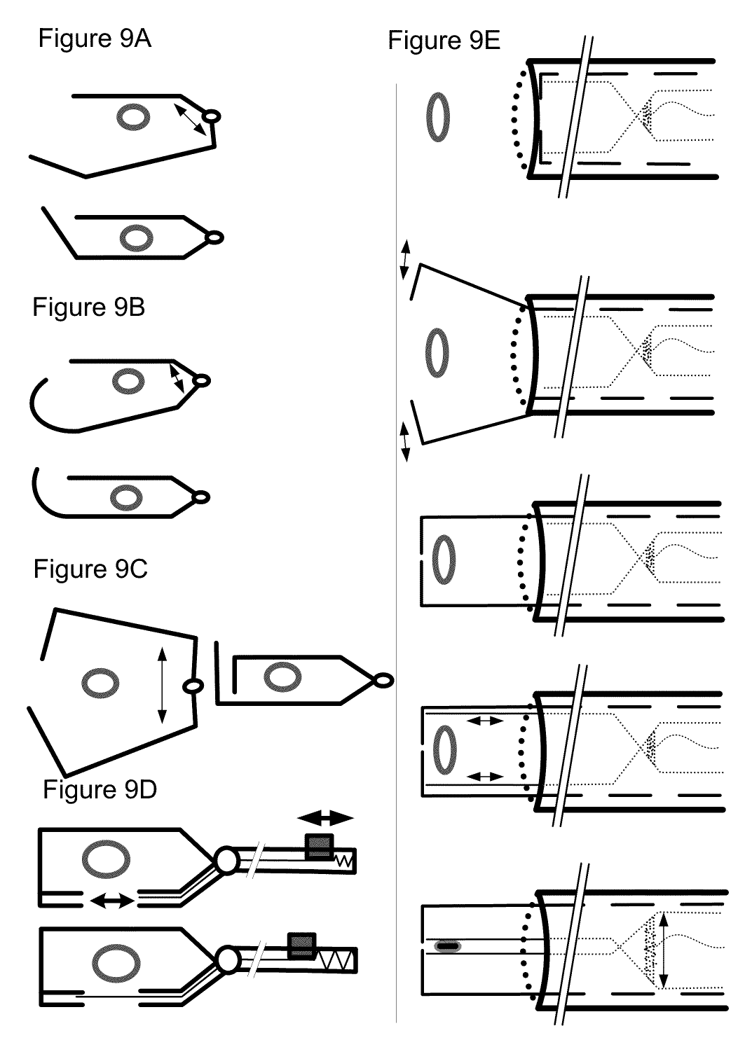

Stabilizing of Blood Vessels: In an embodiment, in addition to the occluding member, one or more stabilizing members can be provided to ensure proper security and stability of the blood vessel during the one or more sets of occlusion and release as described herein. For example FIGS. 9A-E depict cross-sectional views of embodiments of stabilizing members that can be provided. FIG. 9A depicts an angled lip to one jaw in a set of two jaws to entrap the blood vessel and minimize the space in which the blood vessel can slip out of the jaws. Similarly, FIG. 9B depicts a curved lip to one jaw and FIG. 9C depicts lips on both jaws to prevent slippage of the blood vessel. FIG. 9D depicts a spring clip embodiment wherein a spring can be compressed to open a wire attached to the spring in the enclosure of a stabilizing member. Accordingly, the stabilizing member is capable of easily opening, securing a vessel, and closing upon release of the spring. Further, FIG. 9E depicts even sized lips on both jaws of the stabilizing member to ensure stabilization of the blood vessel. Also depicted is a jawed occluding member between the stabilizing members. As depicted, upon stabilization by the stabilizing member, the jawed occluding member can be deployed to compress and occlude the blood vessel. In an embodiment, the one or more stabilizing members can be shaped and designed in any manner suitable in the art to allow for securing the vessel to provide the one or more occlusions and releases of the blood vessel by the occluding members as described.

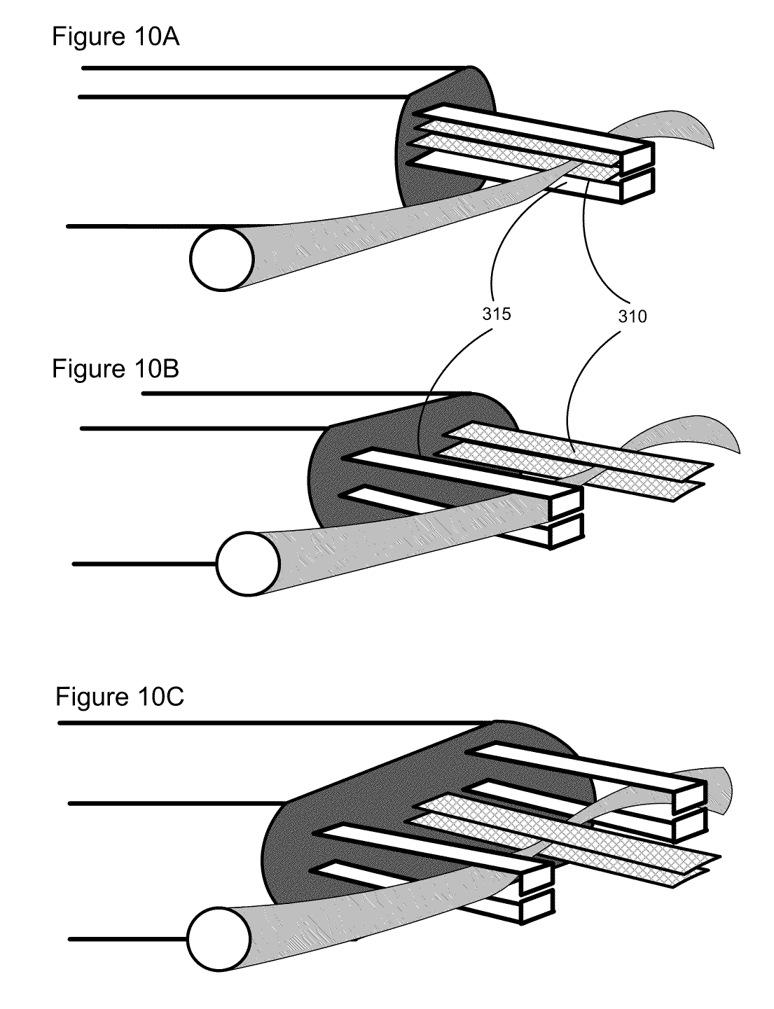

Considering the stabilizing members in more detail, FIGS. 10A-C depict embodiments of orientations of stabilizing members relative to occluding members. FIG. 10A depicts a set of occluding members (310) located within a set of stabilizing members (315). In an embodiment, such an orientation can provide improved access to difficult to reach areas because a reduction of the size of the device can be provided by narrowing the channel that delivers the components. Further, FIG. 10B depicts a set of stabilizing members located beside a set of occluding members. In an embodiment, such an orientation can require a wider opening but provides added stability to the vessel as it is being occluded and released. Even further, FIG. 10C depicts two sets of stabilizing members located around a set of occluding members to ensure that the occlusion and release is completely over the vessel. In an embodiment, providing stabilizing members that are a shorter length than the occluding members can minimize slippage of the blood vessel during multiple occlusions and releases. In an embodiment, one or more of the stabilizing members can be designed to be angled, hooked, enclosed, or any design suitable to ensure security and stability of the vessel as described herein. In an embodiment, the one or more stabilizing members can be oriented in any manner suitable in the art to ensure security and stability of the blood vessel during one or more occlusions and releases as described herein.

Release of Extravascular Occlusions: In an embodiment, release of an extravascular occlusion as described herein can be controlled to a desired amount and duration of occlusion. In an embodiment, release of an extravascular occlusion can be scheduled and/or programmed manually, automatically, by biodegradation, or combinations thereof. In an embodiment, controlling the degree of occlusion release over time can be provided.

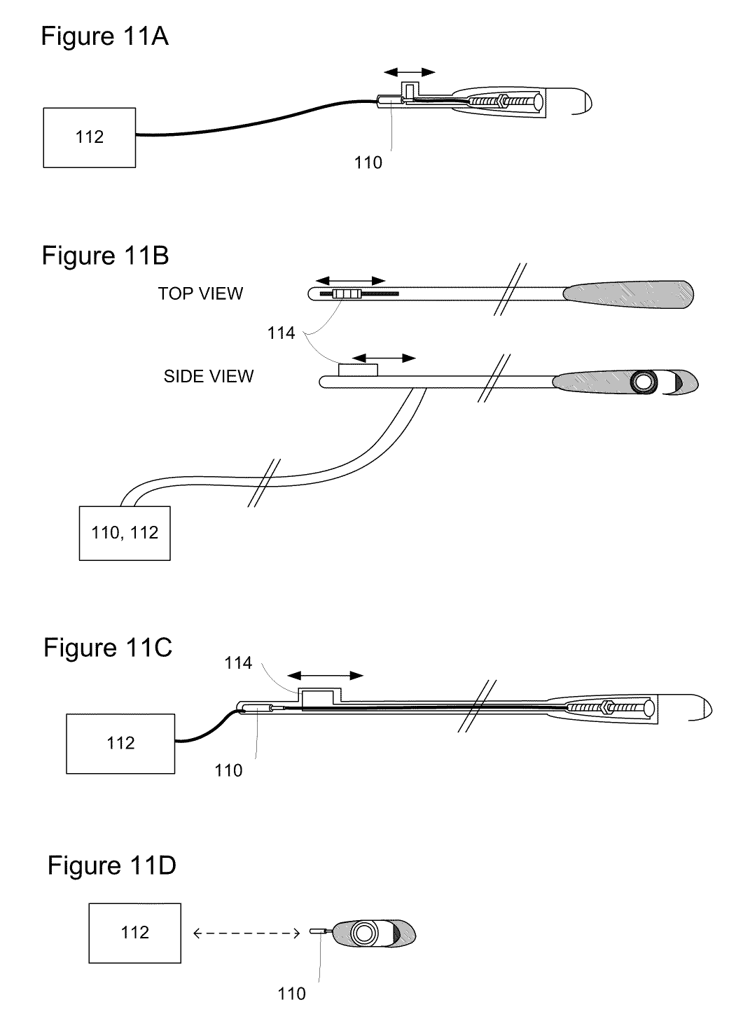

In an embodiment, the compression and decompression of a blood vessel can be timed, scheduled, and/or programmed. Suitable means for controlling the amount and duration of the occlusion can be capable of automatically adjusting the time and compressive force applied to the blood vessel. For example, in an embodiment designed for open surgery as depicted in cross-section view in FIG. 11A, a blood vessel occluder as described herein can be provided with an internal miniaturized motor (110) that is connected by a flexible wire to a controller (112) located remotely. In endoscopic surgery embodiments as depicted in top and side views in FIGS. 11B and 11C, an occluder and the connection between it and the controller (112) can be rigid and at a length suitable enough for such procedures. As depicted, a manual mechanism, such as a switch (114) located on the instrument portion that is outside the body, can provide manual control of a securing mechanism and/or safety release of the occluder. FIG. 11B depicts both the motor (110) and the controller (112) located remotely from the occluder while FIG. 11C depicts the motor (110) located within the occluder while the controller (112) is located remotely. In an embodiment, control of all or a portion of the occluding, securing, and/or safety mechanisms can be provided outside the body as suitable for endoscopic configurations. In an embodiment as depicted in FIG. 11D, the occluder can be provided as an implant with an internal motor (110) that communicates with a remote controller (112).

In an embodiment, communication between the motor and control can be through mechanical, electrical, automated, wired, wireless, and/or any mechanism suitable in the art for the invention as described herein. In an embodiment, the controlling device can schedule timing of complete occlusions and releases. In an embodiment, the device can be programmed to adjust the time intervals or extent of partial occlusions according to a schedule. In an embodiment, any suitable occluding member as described herein and/or that is well known can be adapted to connect to a device that times the compression and decompression of a blood vessel.

In an embodiment, release of extravascular occlusions can be through biodegradation. In an embodiment, the occluding portion of the instrument can be composed of a biodegradable material that releases the occlusion automatically by biodegradation. Suitable biodegradable materials are numerous and well known. In an embodiment, the biodegradable material can be known to biodegrade at a rate that suits a desired time range. For example, an occluding member can be composed of a material known to biodegrade after one hour. Further, in an embodiment, biodegradable occluding members can be composed of different biodegradable materials to allow for biodegradation at various ranges of time. For example, an embodiment of the invention can thus be provided where two biodegradable occluding members can be placed to occlude at the same time but one can occlude for one hour and another can occlude for a day.

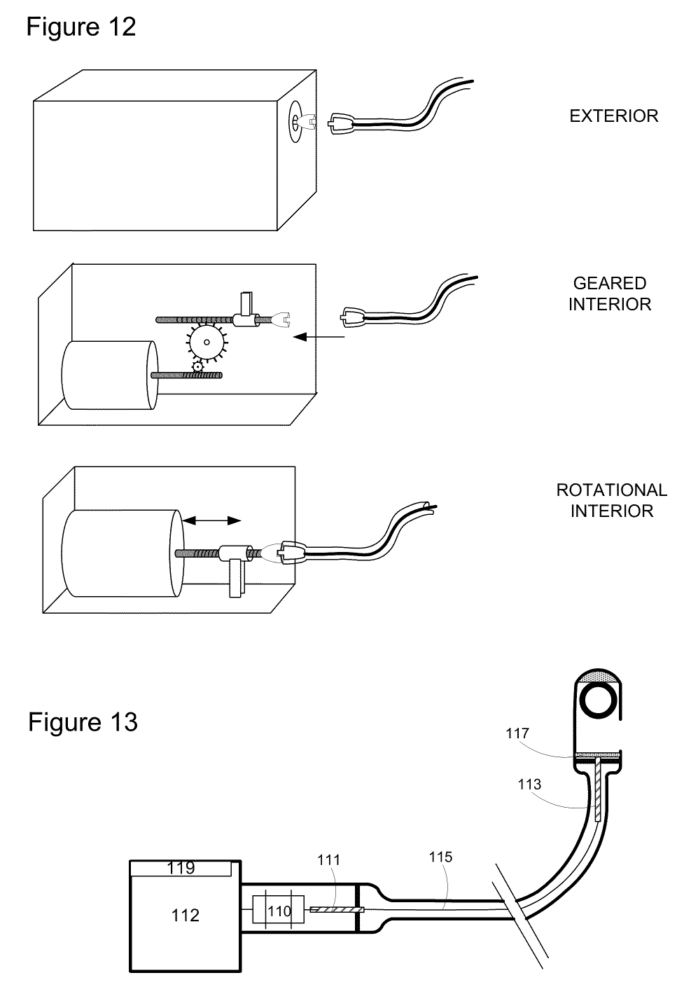

In an embodiment, the amount of release of the occlusion can be controlled by altering the amount of the compressive force applied to the blood vessel. For example, partial occlusions can be provided by applying a compressive force that is known to occlude only a fraction of the blood vessel. Further, in an embodiment, the amount of the occlusion over time can be adjusted by changing the amount of compressive force applied. For example, a complete occlusion can be opened up to various amounts of partial occlusion over time by a device that adjusts the compressive force that is being applied to the blood vessel. FIG. 12 depicts examples of geared and rotationally motored controller boxes that provide controlled force for the invention as described herein. In an embodiment, any other suitable known mechanism to apply controlled force can be provided. Accordingly, as shown in the motorized system for extravascular occlusion of FIG. 13, a controller (112) can transfer energy from a motor (110) to a slide (111) through a rigid untwistable wire (115) to move a corresponding slide (113) attached to a pistoned occluding member to allow for precisely controlled movement of the piston (117). In an embodiment, a display (119) can also be provided to show control, motor, monitoring, and/or any other suitable measurements.

Even further, in an embodiment, biodegradable release of a compressive force can also facilitate adjusting the degree of occlusion over time. In an embodiment, an occlusion can be opened up as an occluding member biodegrades. In an embodiment, the biodegradation can be controlled to release the occlusion in continuous or staggered rates. In an embodiment, a biological response between one or more materials of the occluding member and the body can allow for expansion of the occluding member. In an embodiment, such a bioexpansion can allow for controlling the amount and timing of the occlusion.

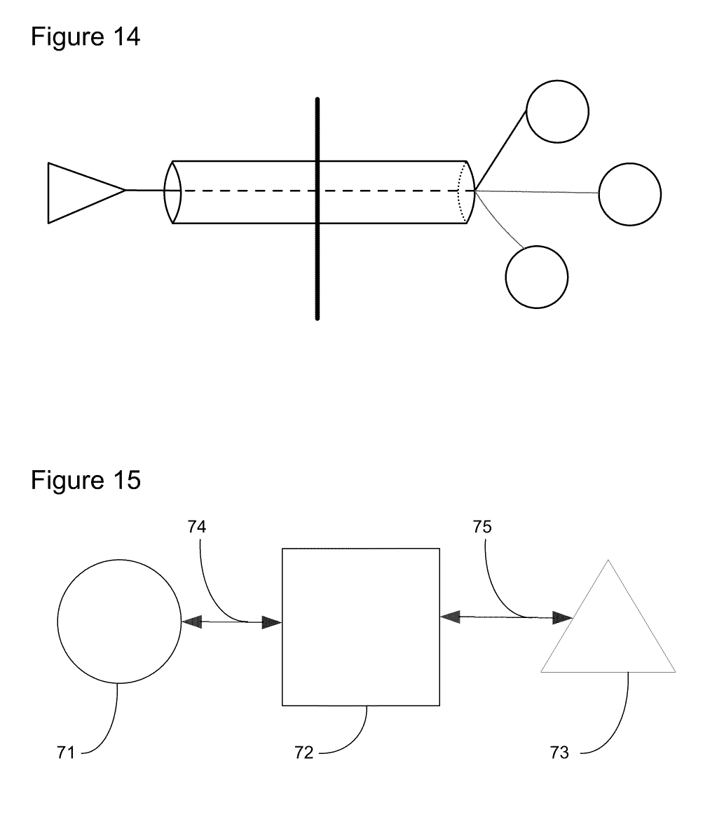

Multiple Extravascular Occlusions: In an embodiment, the invention provides capability of performing multiple extravascular occlusions. In an embodiment, a single occluding member can be positioned around a blood vessel to perform multiple extravascular occlusions. In an embodiment, the occluding member can be affixed to the blood vessel to perform multiple extravascular occlusions. In an embodiment, delivery mechanisms can be detached from the occluding member upon affixing the occluding member to the blood vessel. In an embodiment, several occluding members can be provided in one device to perform multiple extravascular occlusions. For example, a single hollow delivery tubing can house multiple occluding members that can be deployed around multiple blood vessels within the same procedure. Accordingly, in an embodiment, multiple occlusions are provided on multiple blood vessel sites. FIG. 14 depicts a system for performing multiple occlusions on multiple sites simultaneously.

Interventions such as Therapeutic Angiogenesis, Gradual Tissue Death, and Ischemic Conditioning: In an embodiment, an extravascular occlusion as described herein can be particularly advantageous for medical interventions. In an embodiment, one or more occlusions can be beneficial for therapeutic angiogenesis interventions. In an embodiment, one or more occlusions can be beneficial for interventions aiming for gradual tissue death. In an embodiment, one or more occlusions can be beneficial for ischemic conditioning interventions. In an embodiment, monitoring of ischemia resulting from one or more occlusions can be provided.

THERAPEUTIC ANGIOGENESIS: In an embodiment, one or more occlusions as described herein can induce collaterals and/or induce controlled necrosis to benefit a desired therapeutic angiogenesis intervention. Therapeutic angiogenesis is the application of specific compounds in the blood, most of which can result from ischemia and/or occlusion, to inhibit or induce the creation of new blood vessels, or collaterals, in the body in order to combat disease. The presence of blood vessels where there should be none may affect the mechanical properties of a tissue, increasing the likelihood of failure. The absence of blood vessels in a repairing or otherwise metabolically active tissue may retard repair or some other function. Several diseases (e.g. ischemic chronic wounds) are the result of failure or insufficient blood vessel formation and may be treated by a local expansion of blood vessels, thus bringing new nutrients to the site, facilitating repair. Other diseases, such as age-related macular degeneration, may be created by a local expansion of blood vessels, interfering with normal physiological processes.

Formation of vascular collaterals is induced by ischemia and hypoxia of blood vessels. Vascular endothelial growth factor (VEGF) production can be induced in cells that are not receiving enough oxygen. When a cell is deficient in oxygen, it produces the transcription factor Hypoxia Inducible Factor (HIF). HIF stimulates the release of VEGF among other functions including modulation of erythropoeisis. Circulating VEGF then binds to VEGF receptors on endothelial cells and triggers a tyrosine kinase pathway leading to angiogenesis.

The modern clinical application of angiogenesis can be divided into two main areas: anti-angiogenic therapies and pro-angiogenic therapies. Anti-angiogenic therapies can fight cancer and malignancies because tumors, in general, are nutrition- and oxygen-dependent and are thus in need of adequate blood supply. Pro-angiogenic therapies are important in the search of treatment options for cardiovascular diseases. For example, one of the applications of usage of pro-angiogenic methods in humans is using various angiogenic proteins, including several growth factors (e.g. VEGF or fibroblast growth factor 1, FGF-1), some of which can result from ischemia caused by occlusions, for the treatment of coronary artery disease. Clinical research is ongoing to promote therapeutic angiogenesis for a variety of atherosclerotic diseases, including but not limited to coronary heart disease, peripheral arterial disease, and wound healing disorders.

Further, pro-angiogenic therapies by one or more occlusions as described herein can be advantageous due to problems associated with other modes of action, including but not limited to: gene-therapies, protein-therapies (using angiogenic growth factors like FGF-1 or VEGF), and/or cell-based therapies. Problems related to gene therapy include but are not limited to: difficulty integrating the therapeutic DNA (gene) into the genome of target cells; risk of an undesired immune response; potential toxicity, immunogenicity, inflammatory responses and oncogenesis related to viral vectors; and the most commonly occurring disorders in humans such as heart disease, high blood pressure, diabetes, Alzheimer's disease are most likely caused by the combined effects of variations in many genes, and thus injecting a single gene will not be beneficial in these diseases. In contrast, pro-angiogenic protein therapy uses well defined, precisely structured proteins, with previously defined optimal doses of the individual protein for disease states, and with well-known biological effects. On the other hand, an obstacle of protein therapy is the mode of delivery: oral, intravenous, intra-arterial, or intramuscular routes of the protein's administration are not always as effective as desired because the therapeutic protein can be metabolized or cleared before it can enter the target tissue. Also, cell-based pro-angiogenic therapies are still in an early stage of research with many open questions regarding best cell types and dosages to use.

GRADUAL TISSUE DEATH: In an embodiment, one or more occlusions as described herein can be provided for an intervention to achieve gradual tissue death over an extended period of time. For example, sudden and complete occlusion of blood flow for uterine fibroids and/or other benign tissue neoplasms may result in immediate necrosis of tissue and subsequent local and systemic adverse effects. Adverse effects can include inflammation and/or tumor lysis syndrome. The present invention as described herein can allow for controlled release of an occlusion over a period of time to minimize and/or reduce such adverse effects. For example, an occlusion can be controlled to increase an occlusion from 0% occluded to gradually 100% occluded over an extended period of time, such as one to five days, instead of a sudden occlusion.

A benign neoplasm or tumor describes a tumor that lacks all three of the malignant properties of a cancer. Thus, by definition, a benign tumor does not grow in an unlimited, aggressive manner, does not invade surrounding tissues, and does not metastasize. Common examples of benign tumors include moles and uterine fibroids. Some neoplasms which are defined as ‘benign tumors’ because they lack the invasive properties of a cancer, produce negative health effects. Examples of this include tumors which produce a “mass effect” (compression of vital organs such as blood vessels), or “functional” tumors of endocrine tissues, which may overproduce certain hormones (examples include thyroid adenomas, adrenocortical adenomas, and pituitary adenomas). Further, many types of benign tumors have the potential to become malignant and some types, such as teratoma, are notorious for this.

However, treatment of benign neoplasms can require surgery or management of tumor lysis syndrome. Both interventions also require significant management of an inflammatory response. Further, tumor lysis syndrome (TLS) is a group of metabolic complications that can occur after treatment of cancer, usually lymphomas and leukemias, and sometimes even without treatment. These complications are caused by the break-down products of dying cancer cells and include hyperkalemia, hyperphosphatemia, hyperuricemia, hypocalcemia, and acute renal failure. Accordingly, in an embodiment, the present invention as described herein can allow for gradual tissue death over a period of time to provide improved control of the amount of inflammation and/or rate of tumor lysis.

ISCHEMIC CONDITIONING: In an embodiment, one or more occlusions as described herein can be provided for a desired ischemic conditioning intervention. The protective effects of conditioning may be mediated by signal transduction changes to tissues. The current paradigm suggests that nonlethal episodes of ischemia reduce infarct size. Ischemia conditioning has been found to lead to the release of certain substances, such as adenosine and bradykinin. These substances bind to their G-protein-coupled receptors and activate kinase signal transduction cascades. See Id. These kinases converge on the mitochondria, resulting in the opening of the ATP-dependent mitochondrial potassium channel. See Garlid K D et al. “Cardioprotective effect of diazoxide and its interaction with mitochondrial ATP-sensitive K⁺ channels. Possible mechanism of cardioprotection.” Circ Res 81 (1997) 1072-1082. Reactive oxygen species are then released. See Vanden Hoek T L et al., “Reactive oxygen species released from mitochondria during brief hypoxia induce preconditioning in cardiomyocytes.” J Biol Chem 273 (1998) 18092-18098. Thus additional protective signaling kinases can be activated, such as heat shock inducing protein kinase C.

Further, the signaling kinases mediate the transcription of protective distal mediators and effectors, such as inducible nitric oxide synthase, manganese superoxide dismutase, heat-stress proteins and cyclo-oxygenase 2, which manifest 24-72 hours after infarction to provide late protection. Suggested mechanisms of how these signaling transduction pathways mediate protection and ultimately reduce infarct size include maintenance of mitochondrial ATP generation, reduced mitochondrial calcium accumulation, reduced generation of oxidative stress, attenuated apoptotic signaling and inhibition of mitochondrial permeability transition-pore (mPTP) opening. See D M Yellon and J M Downey, “Preconditioning the myocardium: from cellular physiology to clinical cardiology,” Physiol Rev 83 (2003) 1113-1151; Yellon D M, Hausenloy D J, “Realizing the clinical potential of ischemic preconditioning and postconditioning,” Nat Clin Pract Cardiovasc Med. 2(11) (2005) 568-75. It is also possible that alternative protective mechanisms of ischemia conditioning might exist that are independent of signal transduction pathways, such as those mediated by antioxidant and anti-inflammatory mechanisms, and so on.

Ischemia has been shown to produce tolerance to reperfusion damage from subsequent ischemic damage. One physiologic reaction to local ischemia in normal individuals is reactive hyperemia to the previously ischemic tissue. Arterial occlusion results in lack of oxygen (hypoxia) as well as an increase in vasoactive metabolites (including adenosine and prostaglandins) in the tissues downstream from the occlusion. Reduction in oxygen tension in the vascular smooth muscle cells surrounding the arterioles causes relaxation and dilation of the arterioles and thereby decreases vascular resistance. When the occlusion is released, blood flow is normally elevated as a consequence of the reduced vascular resistance.

Perfusion of downstream tissues is further augmented by flow-mediated dilation (FMD) of larger conduit arteries, which acts to prolong the period of increased blood flow. As a consequence of the elevated blood flow induced by reactive hyperemia, downstream conduit vessels undergo luminal shear stress. Endothelial cells lining the arteries are sensitive to shear stress and the stress induces in opening of calcium-activated potassium channels and hyperpolarization of the endothelial cells with resulting calcium entry into the endothelial cells, which then activates endothelial nitric oxide synthase (eNOS). Consequent nitric oxide (NO) elaboration results in vasodilation. Endothelium-derived hyperpolarizing factor (EDHF), which is synthesized by cytochrome epoxygenases and acts through calcium-activated potassium channels, has also been implicated in flow-mediated dilation. Endothelium derived prostaglandins are also thought to be involved in flow-mediated dilation.

Ischemia Preconditioning (IPC) has been found to have remote and systemic protective effects in both human and animal models. Transient limb ischemia (3 cycles of ischemia induced by cuff inflation and deflation) on a contralateral arm provides protection against ischemia-reperfusion (inflation of a 12-cm-wide blood pressure cuff around the upper arm to a pressure of 200 mm Hg for 20 minutes) induced endothelial dysfunction in humans and reduces the extent of myocardial infarction in experimental animals (four cycles of 5 minutes occlusion followed by 5 minutes rest, immediately before occlusion of the left anterior descending (LAD) artery). (Kharbanda R K, et al. Circulation 106 (2002) 2881-2883.)

Recent evidence in a skeletal muscle model has suggested that IPC results in increased functional capillary density, prevention of ischemia/reperfusion induced increases in leukocyte rolling, adhesion, and migration, as well as upregulation of expression of nNOS, iNOS, and eNOS mRNA in ischemia reperfusion injured tissue. (Huang S S, Wei F C, Hung L M. “Ischemic preconditioning attenuates postischemic leukocyte—endothelial cell interactions: role of nitric oxide and protein kinase C” Circulation Journal 70 (8) (2006) 1070-5). Research has also shown that ischemic preconditioning can result in elevations of heat shock proteins, antioxidant enzymes, Mn-superoxide dismutase and glutathione peroxidase, all of which provide protection from free radical damage. (Chen Y S et al. “Protection ‘outside the box’ (skeletal remote preconditioning) in rat model is triggered by free radical pathway” J. Surg. Res. 126 (1) (2005) 92-101).

Although originally described as conferring protection against myocardial damage, preconditioned tissues have been shown to result in ischemia tolerance through reduced energy requirements, altered energy metabolism, better electrolyte homeostasis and genetic re-organization, as well as reperfusion tolerance due to less reactive oxygen species and activated neutrophils released, reduced apoptosis and better microcirculatory perfusion compared to non-preconditioned tissue. (Pasupathy S and Homer-Vanniasinkam S. “Ischaemic preconditioning protects against ischaemia/reperfusion injury: emerging concepts” Eur. J. Vasc. Endovasc. Surg. 29 (2) (2005) 106-15).

In accordance with the novel indication of the present invention, in an embodiment the body's own adaptive responses to induced ischemia or hypoxia are monitored to provide protection against tissue damage and to increase response to therapies. In an embodiment of the invention, duration and frequency of ischemia are adjusted based on monitoring of markers in a target tissue, including but not limited to metabolic, oxygenation, and/or biochemical markers. In an embodiment, supplemental episodes of heat, vibration, drugs, or combinations thereof, are provided based on monitoring of biochemical markers in the target tissue.

In an embodiment, administration of ischemia is chronic, regular or periodic for a period prior to an injurious intervention. For example, the individual patient may schedule a pattern of ischemia, such as for limited periods 5-10 times a day for a period preceding each intervention. In another embodiment, ischemia is administered to the future injury site for a period prior to injury. Depending on responses desired and obtained in the individual patient, the intensity and duration of ischemia can be tuned for optimal responses.

MONITORING: In an embodiment, monitoring of markers can provide measurements to control ischemic preconditioning and postconditioning. In an embodiment, the target tissue has been at least partially damaged prior to inducing ischemia. In an embodiment, ischemia is controlled by postconditioning at the onset of reperfusion to reduce reperfusion injury. In an embodiment, ischemic preconditioning reduces damage to tissue due to a traumatic medical procedure such as surgery, angioplasty, chemotherapy, or radiation. In an embodiment, ischemia and heat can also be similarly adjusted to increase monitored effects of certain therapies, such as drugs and radiotherapy. For example, in an embodiment, neuropathy from chemotherapy and radiotherapy interventions can be reduced or prevented by providing ischemic preconditioning based on monitoring levels of oxygen in a target tissue.

Several studies have indicated that there may be organ-specific biochemical thresholds for dysoxia, and yet heterogeneity of blood flow (or cellular metabolism) within an organ can also lead to different values at different locations within the same organ. For example, for a discussion of pH thresholds related to hepatic dysoxia, see, inter alia, Soller B R et al. “Application of fiberoptic sensors for the study of hepatic dysoxia in swine hemorrhagic shock.” Crit Care Med. 2001 Jul.;29(7):1438-44. Further, overall tissue oxygen sufficiency can be confirmed by near-infrared measurement of cytochrome oxidase and the redox behavior of cytochrome oxidase during an operation is a good predictor of postoperative cerebral outcome. (Kakihana Y, et al., “Redox behavior of cytochrome oxidase and neurological prognosis in 66 patients who underwent thoracic aortic surgery.” Eur J Cardiothorac Surg. 2002 Mar.;21(3):434-9.)

Accordingly, in an embodiment, novel methods and apparatus of this invention allow tissue ischemia to be controlled based on monitoring of these variable biochemical markers by a system for ischemic conditioning. FIG. 15 depicts a system for ischemic conditioning. In an embodiment, a system for ischemic conditioning can include an occluding device (71), a controlling device (72), a sensing device (73), and communication signals (74, 75) between the devices. The occluding device can induce ischemia through one or more episodes of occlusion of blood supply. The occluding device can be controlled by the controlling device via a signal. The sensing device can measure one or more biochemical markers in a target tissue and send information via a signal to the controlling device. Accordingly, the controlling device can control the one or more episodes of occlusion by the occluding device based on monitoring of a signal received from the sensing device.

Considering the occluding device in more detail, ischemia can be induced through one or more episodes of occlusion of blood supply by the occluding device. In an embodiment, the occluding device can include any of the occluding members as described herein. In an embodiment, the occluding device can induce occlusions at a duration and frequency suitable for the size of blood vessels and target tissue being conditioned. For example, in an embodiment, larger coronary arteries can be occluded at a longer duration and slower frequency than smaller blood vessels, such as those found in the brain. In an embodiment, arterial occlusion is desirable in tissues with loose capillary walls as occlusion of the venous system in such tissues can result in unwanted leakage of plasma or blood into the tissue. However, in an another embodiment, to induce ischemia when arterial access for occlusion is unavailable, venous occlusion can be beneficial to prevent or reduce venous blood flow and in turn prevent or reduce arterial blood flow.

The duration and frequency of ischemia varies by therapeutic targets, but both duration and frequency of occlusions can be sustained for longer periods depending on the extent of occlusion. Also, occlusion and release (reactive hyperemia) procedures with different durations and frequencies are implemented depending on individual tolerance and response to therapy.

In an embodiment, duration and frequencies can vary upon a planned intervention schedule so that a desired distal and or contralateral vascular/neuro/neurovascular function is obtained. Occlusion and release is tailored to improve vasoreactivity (increasing the vasodilative capacity) by improving nitric oxide bioavailability (reducing destruction or increasing production). This effect can be seen in the same distal extremity as the occlusion but is also expected to have neurovascular mediated vasodilation of the contralateral extremity as well.

Considering the controlling device and sensing device in more detail, duration and frequency of ischemia and thermal conditioning can be adjusted or stopped by the controlling device based on monitoring of biochemical markers of metabolic activity in the target tissue by the sensing device. For example, if levels of oxygen are monitored as dropping significantly low, the controlling device can alter ischemic episodes to decrease or stop until oxygen levels are monitored to be at a suitable range. Once reaching a desirable range, the ischemic episodes can resume under further monitoring. In an embodiment, a significant enough change in oxygen saturation levels to trigger a conditioning response can be at least 1%. In an embodiment, a significant enough change in oxygen saturation levels to trigger a conditioning response can vary depending on clinical conditions including areas of occlusion, areas of target tissue, duration and frequency of ischemia, and individual tolerance and response to therapy.

Similarly, if levels of other known ischemia-related markers, including but not limited to lactate, pH, carbon dioxide, ATP, ADP, nitric oxide, peroxinitrate, electrolytes, free radicals, Troponin I, Troponin T, CK-MB, BUN, Creatinine, liver transaminases, C-reactive protein, D-dimer, Bradykinin, IL-1, IL-6, IL-8, TNF-α, IFN-γ, TGF-β, IL-1ra, IL-10, iNOS, MnSOD, NF-kappaB, PI3-Kinase/Akt, P38 MAPK, ERK, Caspase 3, PARD, HSP27, VEGF, and combinations thereof, are determined to be changing significantly, the controlling device can adjust ischemic episodes until those levels are monitored to be at a suitable level again. Once reaching a desirable range, the ischemic episodes can resume under further monitoring. In an embodiment, a significant enough change in saturation levels of any biochemical marker to trigger a conditioning response can be at least 1%. In an embodiment, a significant enough change in saturation levels of markers to trigger a conditioning response can vary depending on clinical conditions including areas of occlusion, the particular target tissue, and duration and frequency of ischemia.

Further, if levels of other tissue markers of ischemic conditioning therapy, including but not limited to responses to chemotherapy, radiotherapy, neuropathy, hypertension, chronic conditions, operative outcome, and/or wound healing, are determined to be changing significantly, the controlling device can adjust ischemic episodes until those levels are monitored to be at a suitable level again. Once reaching a desirable range, the ischemic episodes can resume under further monitoring. For example, if tissue markers of chemotherapy induced neuropathy indicate an increase in tissue injury, the frequency of ischemic conditioning treatments can be decreased to prevent or reduce such injury. In an embodiment, measurement of tissue markers of response to ischemic conditioning treatments can include but are not limited to: adenosine, cytochrome oxidase, redox voltage, erythropoietin, bradykinin, opioids, ATP/ADP, and/or related receptors.

Monitoring can be continuous or intermittent, depending on the target tissues and the character of the intervention. For example, monitoring of more distal tissues with slower inherent metabolic rate can be undertaken with more intermittent monitoring than those with high metabolic rates, such as cardiac tissue. Thus, in an embodiment, the desired frequency of monitoring of markers can depend on the extent of the induced ischemia and target tissue areas. In an embodiment, monitoring of tissue markers can provide data to satisfy thresholds of ischemia to adjust the ischemic conditioning protocol in order to prevent or minimize cell injury.

In an embodiment, biochemical markers in the target tissue include levels of lactate, pH, cytochrome oxidase, redox voltage, oxygen, carbon dioxide, ATP, ADP, nitric oxide, peroxinitrate, electrolytes, free radicals, and combinations thereof. In an embodiment, anaerobic conditions during ischemia can change levels of these biochemical markers of metabolic activity in the target tissue. For example, anaerobic respiration can cause lactate levels to increase, pH levels to decrease, oxygen levels to decrease, ATP levels to decrease, and ADP levels to increase. Other biochemical changes can also be measured in the target tissue, such as shifted levels of nitric oxide and peroxinitrate, electrolytes, and free radical redox states. Further, in an embodiment, the induced ischemia is modified and controlled until levels of the biochemical markers are measured to return to desirable ranges.

In an embodiment, biochemical marker measurement can also include thermal markers in the target tissue. Thermal markers can include levels of perfusion, carbon dioxide, external and inherent temperatures, and combinations thereof. Inherent skin temperature means the unaltered temperature of the skin. This is in contrast to an induced skin temperature measurement which measures perfusion by clearance or wash-out of heat induced on the skin. Various methods of recording of inherent skin temperature on a finger tip or palm distal to a noninvasive cuff are disclosed in Naghavi et al., U.S. application Ser. No. 11/563,676 and PCT/US2005/018437 (published as WO2005/118516). The combination of occlusive means and skin temperature monitoring has been termed Digital Temperature Monitoring (DTM) by the present inventor. In an embodiment, the method for monitoring the hyperemic response further includes simultaneously measuring and recording additional physiologic parameters including pulse rate, blood pressure, galvanic response, sweating, core temperature, and/or skin temperature on a thoracic or truncal (abdominal) part.

In an embodiment, tissue markers can be measured noninvasively by suitable well known non-invasive probes in the art, such as, for example, the use of a pulse oximeter for measurement of oxygen saturation. In an embodiment, invasive measurement of biochemical markers can be performed by any suitable well known invasive probes in the art, such as, for example, near-infrared oxygenation probes, visible light oxygenation probes, fluorescent probes for nitric oxide measurement, tissue pH probes, fiber optic redox probes, and sodium and potassium probes for electrolyte measurement. In an embodiment, the probe interface can be adapted to be anchored and/or tethered to the tissue surface being monitored. For example, a probe can be fitted with adhesives, legs, or any suitable component to allow for securing the probe to the surface with minimal to no interference in the monitoring involved. In an embodiment, invasive measurement of biochemical markers can include adapting a sensory mechanism together with a delivery catheter. In an embodiment, the tissue markers can be obtained by blood testing.

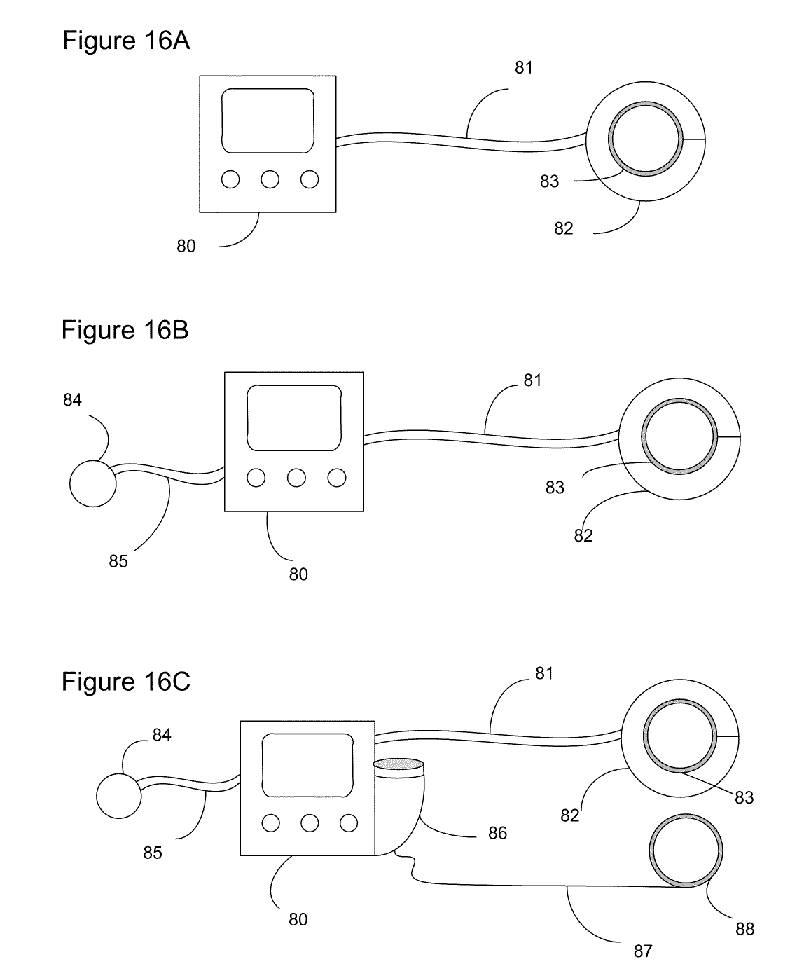

In an embodiment of the invention, a programmable monitor and/or controller is employed to provide ischemia. The device can compress one or more occluding members on one or more blood vessels at a time. FIGS. 16A, 16B, and 16C depict embodiments of different systems for ischemic conditioning. A programmable monitor (80) can be connected by an input (81) to an occluding member (82) (displayed as an extravascular cuff in a cross sectional side view perspective). The programmable monitor can be programmed to alter the compression and/or decompression of the cuff (82) and thus altering compression of an enclosed blood vessel (83). Variables controlled by the programmable monitor (80) include, but are not limited to pressure and time of compression. Thus duration, frequency, and effects of ischemic conditioning can be controlled by the monitor. Any fluids suitable for inflation of the cuff can be used, including but not limited to air, oxygen or water.

In an embodiment, a conditioning pump can also be combined with a distal monitoring sensor (84) connected via an electrical cable (85). The distal monitoring sensor can be capable of taking measurements to monitor biochemical markers of target tissues as described herein. In an embodiment, the pump settings can be capable of electronic adjustment by readings from the sensor. In another embodiment, the conditioning pump can also be combined with a drug infusion system including a reservoir (86) containing one or more pharmaceuticals and an entry line (87) that delivers the medication from the reservoir to a distal artery (88). The reservoir (86) and/or entry line (87) can be attached or separate from the monitor. In an embodiment similar to a diabetes blood sugar monitor, the conditioning pump settings, sensor readings, drug infusion settings, and combinations thereof can communicate electronically with each other and automatically change settings if necessary.

In another embodiment of the invention, apparatus for inducing ischemia in a tissue includes use of one or more releasable “minimally invasive cuffs,” small cuffs adapted to occlude blood supply to a tissue when tightened. The occlusion of blood supply can be partial or complete. In an embodiment the minimally invasive cuffs can include inflatable cuffs designed to be externally situated around at least one blood vessel. In an embodiment, the minimally invasive cuff can be delivered to be externally situated around the at least one blood vessel by a separate delivery device. The apparatus can include a pump that inflates the minimally invasive cuff and thereby occludes the blood supply according to a schedule and can further include one or more distal sensory mechanisms, drug infusion systems, heating mechanisms for intermittent heating of at least one hand or foot, or combinations thereof. The schedule can instruct tightening the cuffs in either a cyclical or sustained manner. The apparatus can be manual in operation or can be automated.

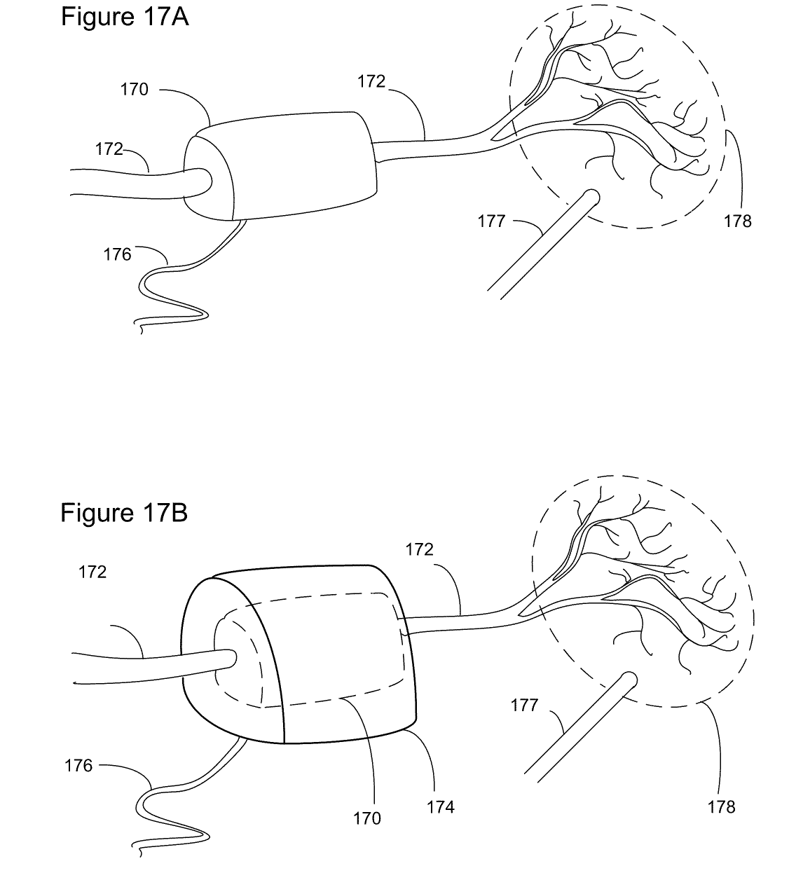

In an embodiment of the invention, ischemia is implemented by minimally invasive cuffs (170) as occluding members that are secured around one or more blood vessels (172) of the patient, as depicted in FIGS. 17A & 17B. In the embodiment depicted in FIG. 17A, the minimally invasive cuff (170) is positioned around the blood vessel (172) yet still allows blood flow to the target tissue area (178). To induce ischemia, an inflating signal can be sent to the minimally invasive cuff (170) via a fluid input (176) and cause the cuff to inflate and compress. In an embodiment, the cuff can inflate outward as depicted in FIG. 17B, the inflated cuff (174) can thus partially or fully occlude blood supply to the target tissue area (178). In an embodiment, the cuff can inflate inward against a rigid outer surface and induce ischemia. In an embodiment, cuff inflation can be both inward and outward. Further, any suitable, well known minimally invasive probe (177) can provide measurement of markers of ischemia, effects of conditioning treatment, blood flow, or combinations thereof in the target tissue (178).

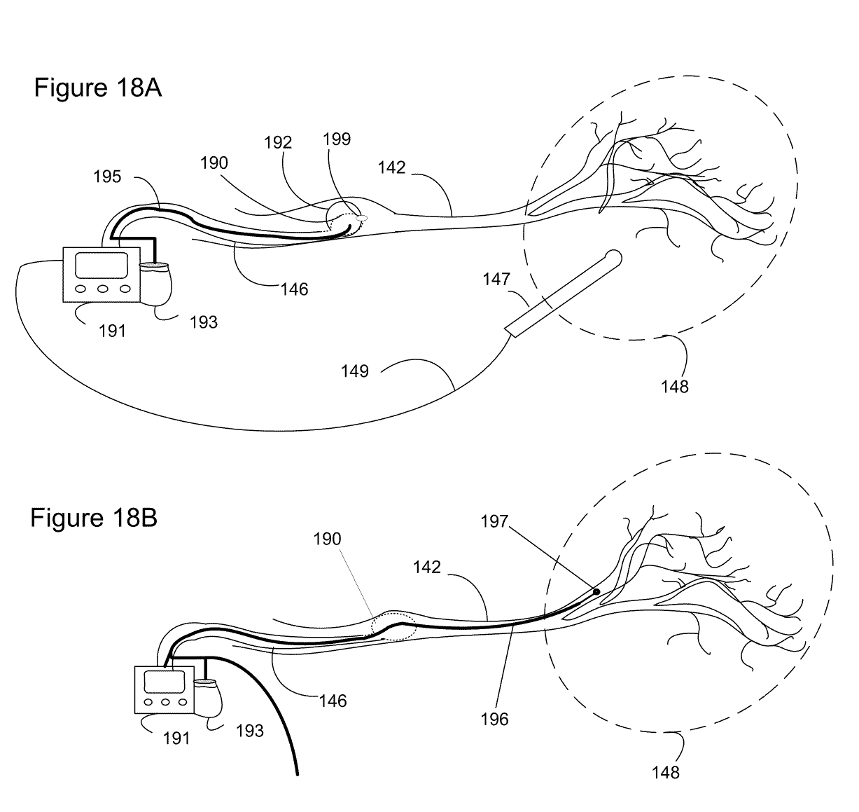

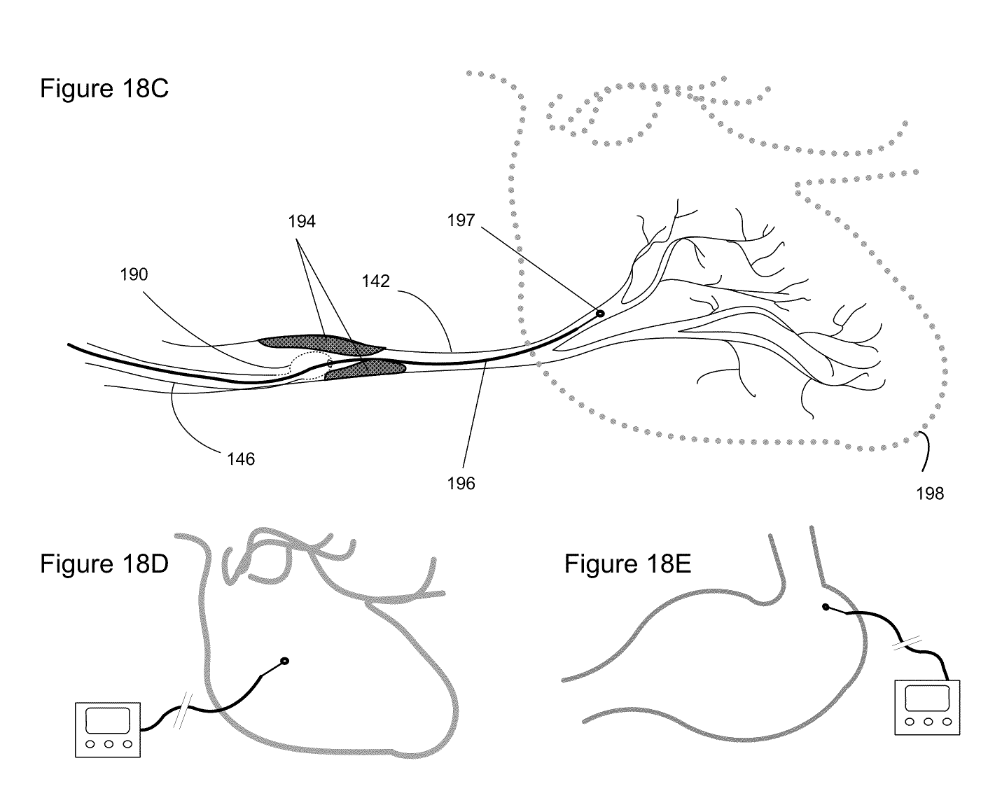

In an embodiment, the apparatus for inducing ischemia includes one or more invasive balloons adapted to occlude blood supply when inflated. Invasive occlusions such as the minimally invasive cuff and the invasive balloon are especially applicable in anticipation of heavy trauma such as in surgery. For example, improved recovery after tumor removals, including kidney, liver, and lung tumors is expected to be obtained in an invasive ischemic conditioning embodiment. The occlusion of blood supply by balloon inflation can be partial or complete. A puncturing device and/or catheter can be separate or used in conjunction with an intravascular balloon. The apparatus can be manual in operation or can be automated. In an embodiment the apparatus includes a programmable monitor for instructing tightening or inflation of the balloon in accordance with a schedule. The apparatus can include a pump that inflates the balloon and thereby occludes the blood supply according to the schedule and can further include one or more distal sensory mechanisms, drug infusion systems, heating mechanisms for intermittent heating of at least one hand or foot, or combinations thereof. The schedule can instruct inflating the balloons in either a cyclical or sustained manner. In an embodiment, invasive measurement of tissue markers can include adapting a sensory mechanism with a catheter well known in the art, such as a guiding wire catheter. A balloon as used for ischemic conditioning can be situated in numerous embodiments, including but not limited to those depicted in FIGS. 18A, 18B, & 18C.

In the embodiment depicted in FIG. 18A, a balloon can be inserted inside a blood vessel (142) in a deflated state (190) and inflated or deflated through a fluid input (146). When in an inflated state (192), the balloon can close one or more locations of the blood vessel sufficient to occlude blood flow to downstream target tissue areas (148). For example, the balloon (190) can be inflated to partially or completely occlude blood flow. Inflation of the balloon (190) can be sustained to hold a partial occlusion or can be adjusted to allow for cyclical occlusions. To release occluded blood supply, the balloon (190) can be deflated. The duration, frequencies, and effects of ischemia from balloons (190) to downstream tissues again vary by therapeutic targets, but are similar to the description provided herein. Further, any suitable, well known invasive probe (147) can provide measurement of markers in the target tissue (148). Materials used for the balloons and fluid inputs are well known in the art and can be any suitable for effecting ischemic conditioning as described.

Further, also as depicted in the embodiment of FIG. 18A, a conditioning pump (191) can be connected to the fluid input (146) and the distal monitoring probe (147) via a cable (149). The conditioning pump can be located internal or external of the body. In an embodiment, the conditioning pump can be capable of controlling inflation and deflation of the balloon via the fluid input. The distal monitoring probe can be capable of taking measurements to monitor biochemical markers of target tissues as described herein. In an embodiment, the pump settings can be capable of electronic adjustment by readings from the probe. In an embodiment, the conditioning pump can also be combined with a drug infusion system including a reservoir (193) containing one or more pharmaceuticals and an entry line (195) that delivers medication from the reservoir to the inside of the balloon lumen (190), where it can be further dispersed to distal blood vessels via a hole (199) in the balloon. The reservoir (193) and/or entry line (195) can be attached or separate from the pump. In an embodiment similar to a diabetes blood sugar monitor, the conditioning pump settings, probe readings, drug infusion settings, and combinations thereof can communicate electronically with each other and automatically change settings if necessary.

In an embodiment, monitoring of the biochemical markers and drug infusion can be administered from inside a balloon lumen via a guidewire catheter adapted with a sensor. Suitable rigid yet flexible guidewire catheters and balloon adaptations for delivering wires to distal tissues are well known in the art. In the embodiment depicted in FIG. 18B, a balloon (190) is again situated in a blood vessel (142) to provide occlusive ischemic conditioning to a target tissue (148). The balloon is connected to a conditioning pump (191) for inflation and deflation via the fluid input (146) of the balloon lumen. The distal sensor (197) is adapted for placement at or near the end of a guidewire (196), both of which can be guided by catheter through a lumen interior of the balloon lumen and into a distal blood vessel of the target tissue (148). Further, drug infusion can also be administered by adapting the guidewire (196) to allow for entry of drugs from a reservoir (193) and infusion of drugs to the target tissue (198) at an end of the guidewire (196). The pump can be located internal or external of the body. The reservoir (193) and/or guidewire (196) are shown as attached to the pump but can be separate in one or more embodiments, provided that monitoring of biochemical markers can still be communicated to the pump to control duration and frequency of ischemia and/or drug infusion.

In the embodiment depicted in FIG. 18C, a balloon catheter (146) can be placed next to a blood vessel obstruction (194) in order to provide a balloon (190) for inducing episodes of ischemia. A guiding wire catheter (196) can be adapted on an end to contain a sensory mechanism (197) capable of detecting biochemical markers. The guiding wire catheter (196) adapted with the sensory mechanism (197) can be inserted through the shaft of the balloon catheter (190), through the balloon (190) via a hole, through the obstruction (194), and into the target tissue area (148). Accordingly, the sensory mechanism (197) can allow for detection of biochemical markers prior to release of the obstructions (194) in the blood vessel. Further, this detection of biochemical markers can then control ischemic episodes induced by the balloon (190) via the balloon catheter (146).

Such an embodiment is especially advantageous in postconditioning to reduce reperfusion injury. With obstructed blood vessels (via obstructions such as plaques or tumors), measurement of biochemical markers in target areas by markers elaborated and detected in the peripheral blood can be too late to avoid reperfusion injury. Thus, in one embodiment of the present invention, an intra-vascular wire adapted with a sensor mechanism is sent through an obstruction to the target area to provide monitoring benefits for controlling ischemic episodes at the onset of reperfusion. Also, adapting a guiding wire catheter with a sensory mechanism to detect biochemical markers can be advantageous in detecting the status of target tissues that are more difficult to detect via other methods, including for example obtaining measurements through the aorta or other coronary ostia, as indicated by the heart target area (198). Suitable catheters and guiding wires for performing such procedures are well known in the art.

Further, the sensory mechanisms can be adapted to be external of the organ or tissue that is targeted for ischemic conditioning. For example, FIGS. 18D and 18E depict embodiments of external sensory mechanisms as used for ischemic conditioning of the heart and stomach, respectively. In an embodiment, external sensory mechanisms can also be used for kidney, liver, lung, or any other tissue suitable for the invention as described herein.

Specifically for the heart, the outflow of venous blood from the heart muscle is via the coronary sinus and blood inflow is via coronary ostia. In an embodiment, a balloon can be placed to occlude and release both inflow blood supply headed to the target tissue via arteries and/or outflow blood draining from the tissue through veins. Similarly, in an embodiment, a sensor can be placed to monitor biochemical markers in both inflow blood supply headed to the target tissue via arteries and/or outflow blood draining from the tissue through veins. Accordingly, in an embodiment, balloon induction of ischemia can be controlled by monitoring of biochemical markers in coronary circulation. Ischemic conditioning using an inflatable balloon to occlude the lumen in the coronary sinus is equivalent to occluding the inflow of coronary arteries. Occlusion of the outflow valve prevents blood from exiting the heart and causes coronary arteries to fill up. Ischemic conditioning with balloons is thus able to be induced in the heart by reducing or preventing exiting blood flow in the coronary sinus. In an embodiment, the coronary sinus can be the location for applications of an invasive balloon and sensory mechanism as used for ischemic conditioning.

Considering the postconditioning applications in more detail, reduction of reperfusion injury can be achieved by both intravascular and extravascular control of reperfusion based on measurement of metabolic markers of ischemia. A dual balloon catheter system that provides intravascular control of reperfusion by pressure sensor measures has been described in U.S. patent application Ser. Nos. 10/499,052, 10/493,779, and 11/689,992. However, these descriptions are limited to intravascular means and pressure measurements. In an embodiment, the present invention provides both intravascular and extravascular means for controlling reperfusion. Further, in an embodiment, improved control of reperfusion flow is provided by measurement and feedback of sensitive metabolic and/or ischemia markers instead of simple pressure measures.

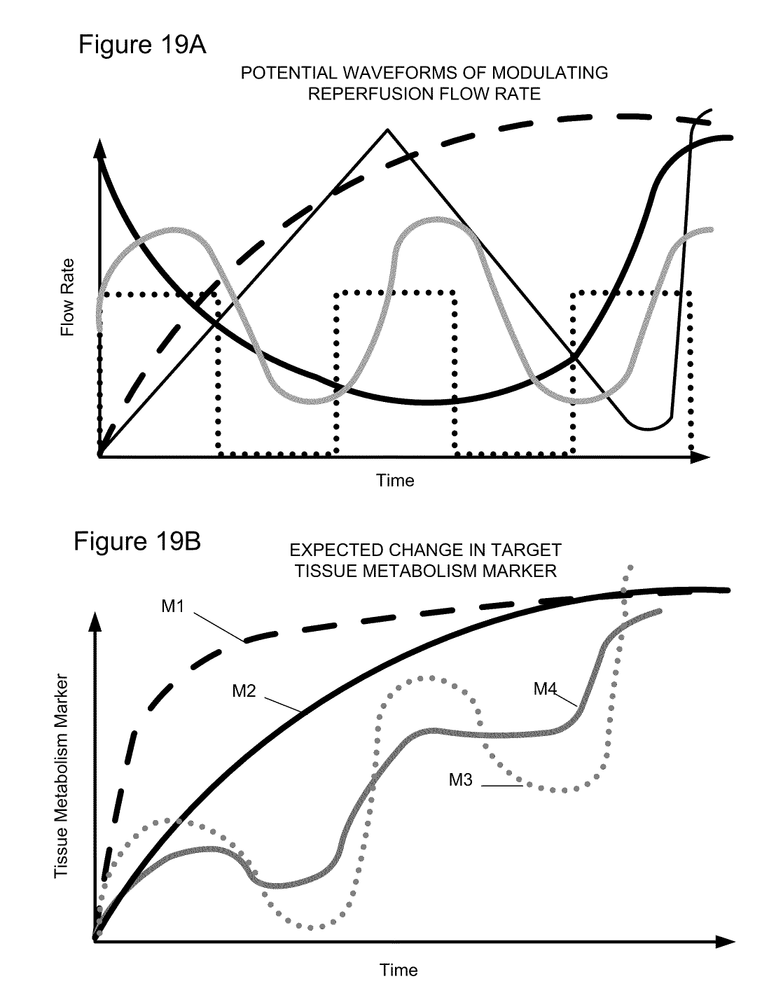

For example, FIG. 19A depicts illustrations of potential modulations to reperfusion flow rate that can be provided. As shown by the depicted variations in flow waveforms, numerous embodiments of linear, sinusoidal, squared, triangle, and/or sawtoothed flow waveforms can be possible by controlling reperfusion. In one or more embodiments, flow starts at zero and then gradually increases linearly; flow starts at zero, gradually increases linearly, then decreases linearly to near zero; flow begins at controlled levels similar to baseline flow (not hyperemic flow) and then gradually decreases linearly; flow begins at controlled levels similar to baseline flow (not hyperemic flow), decreases linearly to near zero, then increases linearly; flow is sinusoidal, never completely reaching zero at its lowest points; or combinations thereof.

Further, FIG. 19B depicts illustrations of expected effects on metabolic markers within a target tissue that can be expected by modulating reperfusion flow rates. In an embodiment, the flow during the controlled reperfusion period is limited and/or determined by a tissue metabolism marker (such as tissue pH, redox state, or level of oxygenated cytochrome oxidase) measured by a probe that is distally located. Reperfusion injury as indicated by rapid changes in tissue metabolic markers can be reduced by monitoring the levels of those changes during modulation of flow rates. As depicted, line M1 shows an exemplary uncontrolled exponential increase in a metabolite whereas M2 shows the expected improved control of the exponential increase of the same metabolite. Similarly, M3 shows an exemplary uncontrolled sinusoidal increase in a metabolite which can show less drastic changes by controlled reperfusion, as indicated by M4. For example, in an embodiment, distal tissue pH can rapidly rise to plateau for uncontrolled reperfusion yet a slow rise curve for controlled reperfusion with the goal of keeping pH within a safe, middle range can be produced until the end of the controlled reperfusion time period. In an embodiment, any non-invasive, minimally invasive, or completely invasive ischemic preconditioning treatment can be combined with the controlled reperfusion of postconditioning to combine the protective effects of both treatments.

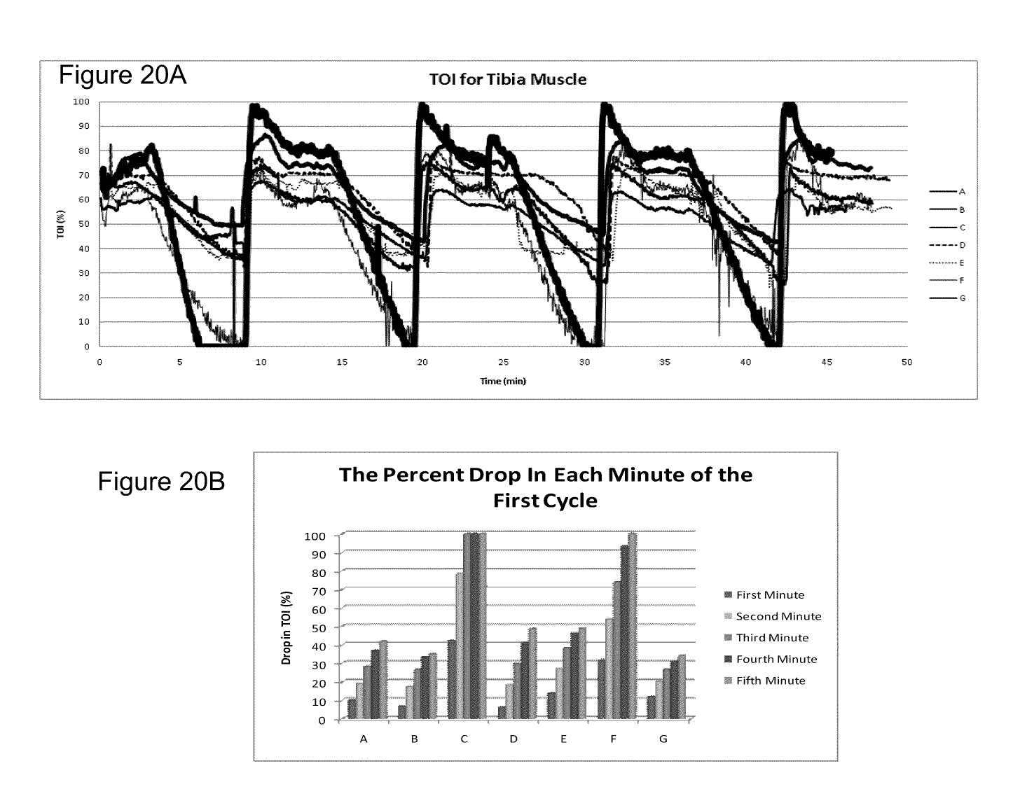

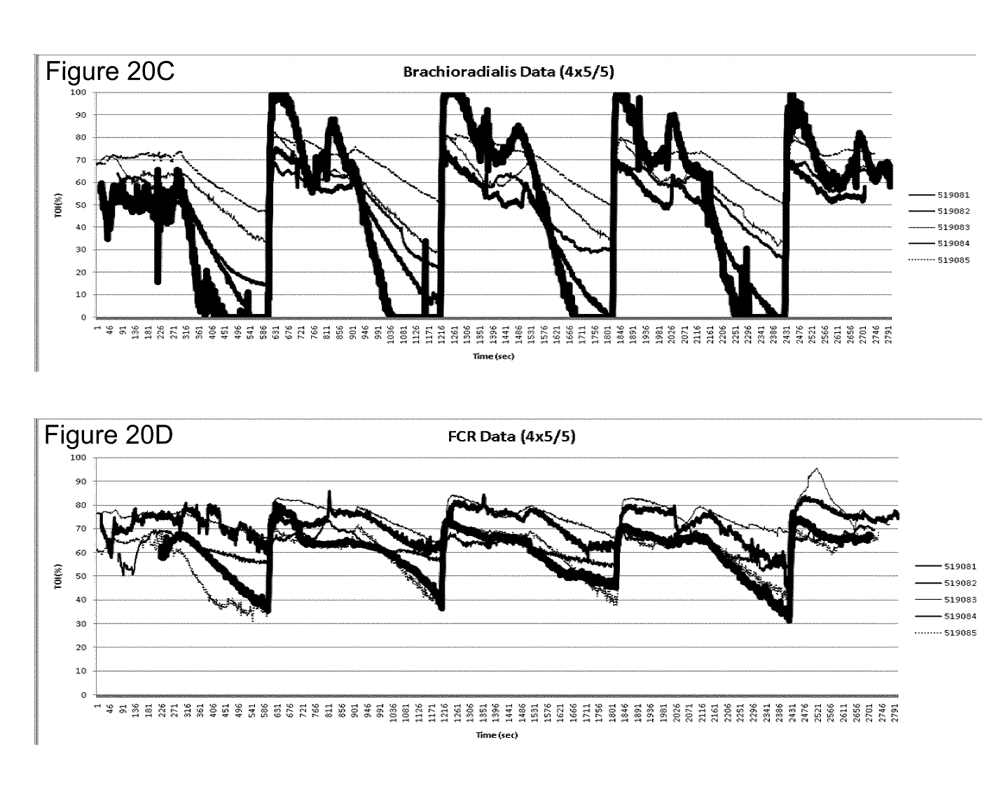

Accordingly, advantages of monitoring during extravascular occlusion include assurance of complete occlusion when needed (e.g. by monitoring pulse using Doppler probes, pulse oximeter, or other well known techniques) and also assurance of adequate levels of ischemia in the target tissue knowing the fact that different tissues experience different levels of ischemia after complete arterial occlusion. As depicted in FIGS. 20A-B, the present inventors have shown that oxygenation can vary among individuals based on a measured response of the vasculature to vascular occlusion utilizing continuous skin monitoring of oxygenation on a muscle distal (downstream) to an occluded arterial flow. A group of seven normal individuals was selected and each was subjected to four consecutive cycles of five minute occlusion followed by five minute release from a cuff placed on the mid thigh. Continuous perfusion status of the downstream tibialis anterior muscle of the lower leg was performed utilizing continuous, real-time, and direct measurement of hemoglobin oxygen saturation in tissue using near infrared (NIR) light to illuminate tissue. NIR measurement of tissue oxygenation is a well known method that analyzes the returned light and can produce a total oxygenation index (TOI), a quantitative measurement of oxygen saturation in the microcirculation of the tissue. As shown in FIG. 20A, there are significant variations in the amount of TOI during the same ischemic conditioning protocol (cycles of cuff occlusion and release) between individuals. FIG. 20B illustrates various reduction rates of TOI (the slope of drop) measured at each minute (1-5) of the first cycle during the same ischemic conditioning protocol. FIG. 20C provides data obtained by the same technique but utilizing occlusion using a cuff placed over the upper arm and thus occluding the brachial artery while measuring the NIRS data over the brachioradialis muscle on several different individuals. FIG. 20D depicts data where the NIRS probe was placed over the flexi carpi radialis (FCR) muscle of the arm.

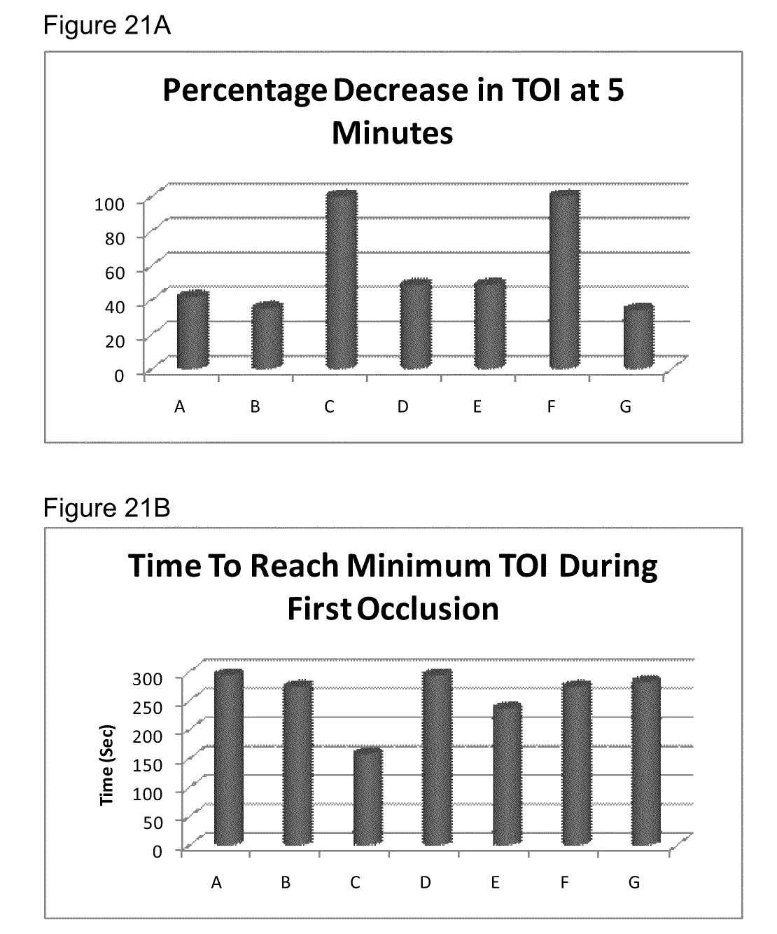

FIGS. 21A and B depict the difference between individuals in reaching minimum TOI (maximum ischemia). As depicted in FIG. 21A, the percentage drop of TOI after five minutes of cuff occlusion varies by 293.7% (100% drop versus 34% drop). Further, FIG. 21B shows the time to reach minimum TOI (maximum ischemia) ranged from 2.6 minutes to 4.9 minutes in this study group. These observations clearly indicate the need for monitoring tissue ischemia during ischemic conditioning, so that ischemic conditioning protocols can be tailored to each individual according to their physiologic characteristics, such as metabolic rate and blood oxygenation status. Accordingly, a proper ischemic conditioning system requires monitoring of tissue ischemia to assure the desired level of ischemia is achieved and maintained for the duration intended.

Combination Therapies: In one embodiment of the invention, at least one ischemic conditioning treatment of induced ischemia or hypoxia and/or application of heat, vibration, and/or counterpulsation are combined with pharmacotherapy including by administration of an anti-hypertensive agent, vasodilating agent, anti-oxidant, anaestheic, and/or anti-inflammatory agent. Multiple compounds are known in each of these categories. Existing vasodilators include for example hydralazine, ACE inhibitors (such as for example enalapril), alpha-beta blockers (such as for example carvedilol), minoxidil, and calcium channel blockers (such as for example nisoldipine, nifedipine, diltiazem and verapamil). New vasodilators such as, for example, oxdralazine are being developed and may be equally suitable. Pharmacotherapy includes agents that increase the local bioavailability of NO. The pharmacotherapy can be administered systemically or locally, such as by iontophoresis.

In another embodiment, at least one ischemic conditioning treatment of induced ischemia or hypoxia, and/or application of heat or vibration, is combined with non-pharmacologic techniques, mostly for regional and transient modulation based on anatomical reflex zones.

These non-pharmacologic techniques may include non-invasive electric, magnetic, or electromagnetic devices. In another embodiment, transient intermittent ischemia and or heating is combined with hand exercises to increase demand and thereby improve nitric oxide bioavailability in the target areas. In an embodiment of the invention, conditioning is enhanced by drugs delivered to affected distal extremities by iontophoresis. The current for driving iontophoresis can be supplied by a regulated power supply in connection with a source of line current or can be supplied by a battery. In an embodiment, the drug is an anesthetic drug. In other embodiments the drug is an anti-inflammatory drug. In other embodiments, the drug is an NO donor. Combinations of drugs can be selected for co-delivery depending on their shared ionic properties.

Intermittent Heating for Protection and Treatment In an alternative embodiment, increased blood flow, enhanced metabolic activity, and anti-oxidant capability is obtained by intermittent heating of the hands and/or feet, or digits thereof. Heat is employed to shift the sympathetic-parasympathetic balance, including through the induced increase in local production of nitric oxide, in order to induce vasodilation and reduced resistance to peripheral blood flow.

In certain embodiments, the heat is provided by a wearable appliance that includes a heating element, a heating controller connected to the heating element, and a source of power for the heating element. As used herein, the term “wearable appliance” includes heatable inserts or pads that are dimensioned for placement in desired anatomical locations, including stand-alone appliances, appliances disposed in garments, and appliances that are used in association with a garment. Appliances that are used in association with a garment include appliances that are worn inside and those that are worn outside of the garment. As used herein, the term “non-wearable” appliance includes fixtures and/or portable devices that are not dimensioned to be attached or carried by an individual during ambulation.

In an embodiment, a wearable heat conditioning appliance can be dimensioned to be worn as mittens, socks or booties, or gloves. The heating applied must be of sufficient magnitude to cause vasodilation. The optimal site for heating, as well as the intensity and duration of heating, can be readily determined for a given individual based on whether or not the desired vasodilation is obtained.

In an embodiment, local administration of heat is chronic, regular or periodic for a period prior to the injurious intervention. For example, the individual patient may schedule a pattern of heating, such as for limited periods 5-10 times a day for a period preceding each intervention. In another embodiment, heat is administered to the future injury site for a period prior to injury. Depending on responses desired and obtained in the individual patient, the intensity and duration of heat can be tuned for optimal responses.

In an embodiment the heating method is conventional such as by electric heating coils or is provided by ultrasound, microwave (MW), radio frequency (RF) energy, and/or other forms of electromagnetic energy such as infrared radiation. In other embodiments, heat is provided by a chemical reaction such as by oxidation of iron. In another embodiment, heat is provided via combustible energy sources such as butane or propane heaters. Power can be delivered through a wearable power supply and cause heat on demand.

In an embodiment ultrasound, microwave (MW) and/or radio frequency (RF) diathermy is employed to generate deep heating up to 2 inches from the skin surface without damage to the skin. The phrase “diathermy” means the controlled production of deep heating beneath the skin in the subcutaneous tissues, deep muscles and joints for therapeutic purposes. Current diathermy devices on the market generate deep heating by using radio (high) frequency, microwave or ultrasonic energy.

Ultrasound diathermy applies high-frequency acoustic vibration to tissues thereby generating heat. Current ultrasonic diathermy devices operate in a frequency range of 0.8 to 1 MH Z. MW diathermy applies a strong electrical field with comparatively low magnetic-field energy to induce intra-molecular vibration of highly polar molecules within the treated tissue to generate a thermal effect. Microwave diathermy is assigned 915 MH Z and 2450 MH Z as operating frequencies (these are also microwave oven frequencies). RF diathermy involves application of shortwave length, high-frequency electromagnetic fields. Radio frequency (RF) diathermy is assigned an operating frequency of 27.12 MH Z (short wave) by the Federal Communications Commission. The electromagnetic field can be perpendicular or longitudinal in orientation. Although perpendicular electromagnetic field devices have been historically utilized in medical RF diathermy devices, devices able to deliver low-energy longitudinal fields are also available (i.e. Selicor Brand Selitherm devices) and are applicable to the present invention.

The present informal position of the Food and Drug Administration is that a diathermy device should be capable of producing heat in tissue from a minimum of 104° F. to a maximum of 114° F. at a depth of two inches in not more than 20 minutes. RF heating can be done by dielectric or inductive methods and the physical configuration of the device is designed in accordance with electrical engineering principals depending on the ultrasound, MW or RF method desired.

In an embodiment of the invention, the heating is provided by Far Infrared Radiation (FIR). Commercially available versions of such elements able to provide heat to subcutaneous tissue include, for example, FIR Radiant Heating elements. (Challenge Carbon Technology Co., Taiwan). Such elements are suited for FIR heated clothing due to their flat form and foldable, durable and washable properties. The elements as provided for use in clothing may include batteries (such as, for example, lithium-ion batteries), temperature controllers and OCP (Over-Charge Protector) integrated in one controller that provides for rapid heat up according to set upper levels.

Counterpulsation: Alternatively or in addition to other conditioning treatments, in one embodiment counterpulsation sufficient to diminish ischemic cardiomyopathy is applied to at least one distal extremity of the patient. The counterpulsation may be performed by any suitable regimen to increase cardiac output by decreasing the afterload that the heart has to pump against and increasing the preload that fills the heart. For example, a regimen can include repetitions using series of pneumatic stockings or cuffs on legs that are connected to telemetry monitors to monitor heart rate and rhythm while the cuffs are timed to inflate at the beginning of diastole and deflate at the beginning of systole based on an electrocardiogram.

Clinical Indications for Ischemic Conditioning: Several clinical indications share the commonalities of anticipated injury, stress, inflammation, and toxicity to tissue. In an embodiment of the present invention, the inventors believe that the increase in perfusion, relaxation of smooth muscle cells, vasodilation, anti-inflammatory, and anti-oxidant effects of ischemic conditioning empowers the innate ability of tissue against anticipated insults and stressors. For example, effects of ischemic conditioning as described herein are believed to benefit treatment of neuropathy by administering ischemic conditioning as described herein. Chemotherapy or diabetes induced neuropathy can be anticipated and is believed to be reduced or prevented by ischemic conditioning increasing the innate oxygenation and strength of nerve cells against injury. In an embodiment, enhanced treatment of pain and reduction of pain can be expected from ischemic conditioning. Also, ischemic conditioning as described herein of vascular tissues is believed to systemically prevent or reduce cardiovascular and neurovascular injuries such as those associated with angina, hypertension, and transient ischemic attacks, or TIAs. Further, efficacy of immune suppressant therapies that lower the body's normal immune response are believed to be enhanced by the anti-inflammatory effects of ischemic conditioning as described herein. For example, in an embodiment, ischemic conditioning can be expected to protect against anticipated stressors, including but not limited to endotoxins, such as LPS, responding to stress and/or infection.

Ischemic Conditioning to Reduce Perioperative Complications: Consideration of perioperative complications is critical before, during, and after a surgical procedure. For example, cardiovascular disease and pulmonary disease are both associated with poor outcome of surgery. Intraoperative complications during surgery, e.g. hemorrhage or perforation of organs, can have lethal sequelae. Numerous postoperative complications also exist. For example, local infection of the operative field is possible. Acute respiratory distress syndrome (ARDS) and hypostatic pneumonia due to shallow inspirations frequently occur especially in patients recovering from abdominal surgery, including but not limited to valvular surgery, lung resection, esophagus resection, and/or vascular surgery. Cerebrovascular accidents also occur at a higher rate during the postoperative period.

Accordingly, any protective effect that can be provided to the anticipated tissue of surgery can be of benefit. In an embodiment, the present invention as described herein aims to strengthen tissues under perioperative conditions. In one embodiment of the present invention, intermittent transient ischemia is induced to one or more tissues or organs of anticipated surgery in a patient. The intermittent transient ischemia stimulates and conditions the vasculature and thereby prevents or reduces perioperative complications.

For example, kidney damage can be reduced and/or prevented by ischemic conditioning of the kidney. In an embodiment, contrast-induced nephropathy can be reduced and/or prevented upon ischemic conditioning of a kidney prior to injection of a damaging contrast dye. In an embodiment, ischemic conditioning can reduce or prevent acute kidney injury during and after major surgeries such as cardiac bypass, vascular surgeries, and aortic aneurysm surgery.

Thus, in an embodiment, multiple separate ischemic conditioning treatments can be scheduled in any suitable manner prior to, during, and/or after surgery as described herein, including but not limited to: several times daily, frequently over extended periods of time, based on monitoring and/or assessments of specific interventions and/or treatment resistance. Further, in an embodiment, one or more of the ischemic conditioning treatments can be administered remotely from the operative tissue and provide a systemic effect. For example, minimally invasive cuffs can perform ischemic conditioning in an extremity, such as an arm or leg, to improve postoperative healing from an incision in a part of the body that is difficult to access for occlusion, like the back, chest, or torso. In an embodiment, any extravascular occlusion can be performed intra-operatively on a blood vessel remotely located from the target organ or tissue that is operated on to provide invasive, remote ischemic preconditioning. For example, occlusion of both iliac arteries to elicit preconditioning protection on the heart and kidneys has recently been published. See “Remote Ischemic Preconditioning Reduces Myocardial and Renal Injury After Elective Abdominal Aortic Aneurysm Repair”, Ali, et al., Circulation, 2007].

Ischemic Conditioning to Minimize Postoperative Complications of Cardiothoracic, Vascular, and/or Gastrointestinal Surgeries: Several cardiothoracic, vascular, and/or abdominal interventions can particularly benefit from ischemic conditioning. These interventions can be very complex and take extensive amounts of time to perform. For example, esophageal, colon, and lung surgeries can have significant postoperative complications. The principal objective of an esophagectomy is to remove the esophagus. In most cases, the stomach is transplanted into the neck and the stomach takes the place originally occupied by the esophagus. In some cases, the removed esophagus is replaced by another hollow structure, such as the patient's colon. This procedure is normally done to remove cancerous tumors from the body, but has significant postoperative morbidity and mortality. A significant postoperative complication from the ischemic and hypoxic conditions of esophagectomy is esophageal anastomotic leak.

In medicine, anastomosis is the surgical connection of two structures, such as connections between blood vessels or between other tubular structures such as loops of intestine. Surgical anastomosis occurs when a segment of the tubular structure is resected and the two remaining ends are sewn or stapled together. An anastamotic leak often results from breakdown of a suture line in injured tissue in a surgical anastomosis with leakage of gastric or intestinal fluid, following surgical intervention involving anastomosis of gastrointestinal or bowel structures. Many other complications result from the ischemic and hypoxic conditions of the procedure. For example, pneumonia, Acute Respiratory Distress Syndrome (ARDS), atelectasis, deep vein thrombosis, pulmonary emboli, gastric necrosis, cardiac arrhythmias, myocardial infarction, prolonged ileus, wound infection, sepsis, bleeding, stenosis, and/or anastomotic stricture are all established postoperative complications.

Accordingly, any protective effect that can be provided to the esophageal tissue of surgery can be of benefit. In an embodiment, the present invention as described herein aims to strengthen the esophageal tissues that remain in the body under perioperative conditions. In one embodiment of the present invention, intermittent transient ischemia is induced through gastric arteries to one or more esophageal tissues or gastrointestinal organs of anticipated surgery in a patient. The intermittent transient ischemia stimulates and conditions the target tissue and thereby prevents or reduces perioperative complications of that tissue. Thus, in an embodiment, multiple separate ischemic conditioning treatments can be scheduled in any suitable manner prior to, during, and/or after an esophagectomy surgery as described herein, including but not limited to: several times daily, frequently over extended periods of time, based on monitoring and/or assessments of specific interventions and/or treatment resistance. For example, ischemic conditioning of the stomach and esophagus prior to an esophagectomy can be provided by occluding and releasing an accessible gastric artery (e.g. left gastric artery) at a schedule based on monitoring of ischemia at the tip of the fundus. Similarly, postoperative complications in other surgical interventions can be minimized with ischemic conditioning. For example, a lung resection also often results in systemic ARDS and pneumonia that could be reduced by ischemic conditioning from the pulmonary artery. Further, colon resections can be particularly easily adaptable to ischemic conditioning treatments as the entire colon could be conditioned at once by inducing intermittent ischemia through the mesenteric arterial tree. Even further, postoperative complications from resections of other intestinal interventions, e.g. complex supercharged jejunum procedures, can be reduced by ischemic conditioning as described herein.

Tissue Conditioning for Transplants, Implants, and Grafting: In an embodiment, the invention as described herein can be particularly suited to apply ischemic conditioning to reduce complications and/or improve outcomes for organ or tissue transplants, implants, and/or grafts. A transplant is the moving of a whole or partial organ from one body to another or from a donor site on the patient's own body, for the purpose of replacing the recipient's damaged or failing organ with a working one from a donor site. Donor tissue can be living or deceased. Generally, transplants can be categorized into organ transplants and tissue transplants. Examples of organs that can be transplanted are the heart, kidneys, liver, lungs, pancreas, and intestine. Examples of tissues include bones, tendons, cornea, heart valves, veins, and skin. Further, in medicine, grafting is a sensitive surgical procedure to transplant tissue without a blood supply. The implanted tissue must obtain a blood supply from the new vascular bed or otherwise die. The term is most commonly applied to skin grafting, however many tissues can be grafted, including but not limited to: skin, bone, nerves, tendons, and cornea.

Animal research has shown that ischemic preconditioning protects grafts from subsequent long-term cold preservation-reperfusion injury. See e.g. Yin et al., “Protective effect of ischemic preconditioning on liver preservation-reperfusion injury in rats,” Transplantation. 1998 Jul. 27;66(2):152-7 [a rat liver transplantation model]. Further, in a recent 2007 publication, remote ischemic preconditioning has been shown to clinically benefit patients undergoing coronary artery bypass graft (CABG) interventions. Hausenloy et al., “Effect of remote ischaemic preconditioning on myocardial injury in patients undergoing coronary artery bypass graft surgery: a randomised controlled trial,” Lancet 2007; 370: 575-79.

The present inventors believe ischemic conditioning protocols can be improved for transplants, implants, and/or grafts. For example, ischemic conditioning of donor cells prior to the intervention is believed to strengthen tissue and improve their survival after transplantation. In an embodiment, donor tissue therapies such as hypothermia and/or preservatives can be enhanced by ischemic conditioning of that donor tissue. In an embodiment, sensitive cardioplegia procedures in particular can benefit from ischemic conditioning and strengthening of the cardiac tissue. In an embodiment, kidney transplants can be easily treated as they can require conditioning through only one artery. In an embodiment, duration and frequency of multiple administrations of ischemic conditioning can be optimized for a planned intervention. In an embodiment, direct and/or remote ischemic conditioning of donor cells can be provided prior to a transplant, implant, or graft. Further, ischemic conditioning of the recipient tissue can also be provided. Considering skin grafts in particular, in an embodiment, the donor and/or host tissue of a skin graft or skin flap can undergo superficially pressured ischemic conditioning according to an optimized protocol to improve outcomes of grafting.

1. An instrument for inducing ischemic conditioning by controlled extravascular occlusion, comprising:
at least one occluding member adapted to partially or completely surround at least one blood vessel, and
a controller operably connected to the at least one occluding member, wherein the controller is adapted to remotely control occlusion and release of the blood vessel by the at least one occluding member.

2. The instrument of claim 1, wherein the controller is operably connected to the at least one occluding member by a wireless connection.

3. The instrument of claim 1, further comprising one ore more sensors operably connected to the controller and adapted to detect sensed markers of ischemia, blood flow, or metabolism, and combinations thereof.

4. The instrument of claim 3 wherein the sensed markers of ischemia include one or more of tissue oxygenation, and levels of hemoglobin, and the sensed markers of metabolism include one ore more of lactate, pH, oxygen, carbon dioxide, ATP, ADP, adenosine, cytochrome oxidase, redox voltage, erythropoietin, bradykinin, and opioids.

5. The instrument of claim 3 wherein the controller is a programmable controller that remotely controls extent, duration, frequency, and combinations thereof, of occlusion and release of a flow of blood through the luminal tissue based on feedback from the one or more sensors.

6. The instrument of claim 1 wherein the occluding member is a clamp that is pistoned, jawed, coiled, inflatable, steerable, ringed, or capable of timed release, and combinations thereof.

7. The instrument of claim 5 wherein the programmable controller is a mechanical controller or an electromagnetic controller, or a combination thereof.

8. The instrument of claim 6 wherein the clamp is biodegradable.

9. The instrument of claim 1 wherein the occluding member has the capability to be guided to, positioned on, and tightened around a luminal tissue that is not a blood vessel.

10. The instrument of claim 5 wherein the controller is implanted subcutaneously.

11. The instrument of claim 1 wherein the occluding member is capable of surrounding more than one blood vessel at the same time.

12. The instrument of claim 1 wherein the occlusion and release by the occluding member is manual, automated, or combinations thereof.

13. The instrument of claim 1, wherein the occluding member is an inflatable clamp and the instrument further comprises a pump operably connected to the occluding member to inflate the inflatable clamp to sustain partial or complete occlusion of the blood vessel.

14. The instrument of claim 1 wherein the clamp is adapted and programmed to sustain partial occlusion of a blood supply or alternatively completely occlude the blood supply based on the sensed markers.

15. An instrument for inducing ischemic conditioning in a tissue by controlled intravascular occlusion of one or more blood vessels feeding the tissue, comprising:
an occluding member;
a sensor disposed distal to the occluding member and adapted to detect one or more of: markers of ischemia or metabolism, and combinations thereof in the tissue; and
a programmable controller operably connected to the occluding member and the sensor and adapted to control the occlusion and release of the blood vessel by the occluding member based on the sensed markers.

16. The instrument of claim 15, wherein the occluding member is a inflatable balloon and further comprising a puncturing device, an angioplasty balloon catheter, or combinations thereof, attached to the occluding member.

17. The instrument of claim 15, wherein the occluding member is adapted to reduce or prevent exiting venous blood outflow.

18. The instrument of claim 15, wherein the occlusion and release by the occluding member is manual, automated, or combinations thereof.

19. The instrument of claim 15, wherein the occluding member is adapted to induce controlled reperfusion.

20. The instrument of claim 15, wherein the occluding member is adapted to sustain controlled reperfusion according to a schedule, wherein the schedule is selected from one or more of: a sinusoidal schedule, a linear schedule, or an on and off schedule with an off time of less than 5 seconds.

21. The instrument of claim 15, further comprising a blood flow sensor disposed distal to the occluding member.

22. The instrument of claim 15, further comprising a pump operably connected to the programmable controller and adapted to occlude the occluding member based on the sensed markers.

23. The instrument of claim 15, wherein the sensed markers of ischemia include one or more of tissue oxygenation, and levels of hemoglobin, and the sensed markers of metabolism include one ore more of lactate, pH, oxygen, carbon dioxide, ATP, ADP, adenosine, cytochrome oxidase, redox voltage, erythropoietin, bradykinin, and opioids.

24. A method for ischemic conditioning in a patient using an extravascular occlusion, comprising:
guiding an occluding instrument having at least one interior space to a blood vessel of the patient,
positioning the occluding instrument around the blood vessel,
compressing the blood vessel with the occluding instrument to induce at least a partial occlusion,
decompressing the blood vessel to at least a partial release, and
remotely controlling the occlusion and decompressing by the occluding instrument.

25. The method of claim 24, further comprising a step of sensing markers of ischemia, blood flow, or metabolism, and combinations thereof in tissue perfused by the blood vessel.

26. The method of claim 25, further comprising remotely controlling an extent, duration, frequency, or combinations thereof of occlusion and decompressing based on the sensed markers.

27. The method of claim 25 wherein the sensed markers of ischemia include one or more of tissue oxygenation, and levels of hemoglobin, and the sensed markers of metabolism include one ore more of lactate, pH, oxygen, carbon dioxide, ATP, ADP, adenosine, cytochrome oxidase, redox voltage, erythropoietin, bradykinin, and opioids.

28. The method of any of claims 24-27 wherein the ischemic conditioning is adapted to one or more of inducing collaterals or angiogenesis, inducing necrosis, reducing reperfusion injury, or combinations thereof.

29. The method of claim 24 wherein the occlusion is extraluminal and the instrument is guided to, positioned around, and compresses a lumen of the body that is not a blood vessel.

30. The method of claim 24 wherein the ischemic conditioning is administered to reduce complications of cardiothoracic surgery, vascular surgery, gastrointestinal surgery, use of contrast dye, transplants, implants, or grafting, and combinations thereof.

31. The method of claim 24 wherein more than one blood vessel are occluded at the same time by the instrument.

32. A method for intravascular occlusion in a patient comprising:
guiding an instrument to a blood vessel in the patient,
positioning the instrument to contact the blood vessel at an occlusion site,
causing ischemia by interrupting blood flow to a tissue distal to the occlusion site,
monitoring markers of ischemia and/or metabolism in the tissue distal to the occlusion site, and
adjusting the ischemia based on the monitoring results.

33. A method for preventing, minimizing, or reducing reperfusion injury by controlling reperfusion to a target tissue based on measurements of metabolic markers of ischemia in the target tissue.

34. The method of claim 33, wherein the controlled reperfusion is effected by modulations of blood flow, including waveforms of flow rate that are linear, sinusoidal, squared, triangle, sawtoothed, or combinations thereof.

35. The method of claim 33 wherein the target tissue that is monitored is distal to the site of reperfusion.

36. The method of claim 33, further comprising administering a preconditioning procedure prior to controlling reperfusion.

37. The method of claim 33, wherein reperfusion injury is prevented, minimized, or reduced without measurements of the target tissue, but the reperfusion flow rate is maintained below uncontrolled, hyperemic levels without reaching a no-flow state.
